SEQUENCE LISTING (28 sequences)

SEQ ID NO: 1 - protein, 422 aa, Jeotgalicoccus sp. ATCC 8456
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 2 - DNA, 1302 bp, Artificial Sequence
  Codon-optimised expression construct
atgggttggtctcacccacaattcgaaaagcatatggctactttgaagagagataagggtttggataacaccttgaaggttttgaagcaaggttacttgtacaccaccaatcaaagaaacagattgaacacctccgttttccaaacaaaagctttgggtggtaagccattcgttgttgttactggtaaagaaggtgctgaaatgttctacaacaatgacgttgttcaaagagaaggtatgttgccaaagagaattgtcaacactttgtttggtaagggtgccattcatactgttgatggtaagaaacacgttgacagaaaggctttgttcatgtctttgatgactgaaggtaacttgaactacgtcagagaattgactagaactttgtggcatgctaacacccaaagaatggaatctatggatgaagtcaacatctacagagaatccatcgttttgttgaccaaggttggtactagatgggctggtgttcaagctccaccagaagatattgaaagaattgctaccgatatggatattatgatcgactcctttagagctttaggtggtgcttttaaaggttacaaggcttccaaagaagccagaagaagagttgaagattggttggaagaacaaatcatcgaaaccagaaagggtaacattcatccacctgaaggtactgccttgtatgaatttgctcattgggaagattacttgggtaacccaatggattctagaacctgtgctattgatttgatgaacaccttcagaccattgatcgccattaacagatttgtttctttcggtttacacgccatgaacgaaaacccaattaccagagaaaagatcaagtccgaaccagattacgcttacaagtttgctcaagaagttagaagatattacccattcgtcccatttttgccaggtaaagctaaggttgatattgacttccaaggtgtcactattccagctggtgttggtttggctttggatgtttatggtactacccatgatgaatccttgtgggatgatccaaatgaattcagaccagaaagattcgaaacttgggatggttctccattcgatttgattccacaaggtggtggtgattactggactaatcatagatgtgccggtgaatggattaccgttattatcatggaagaaaccatgaagtactttgccgaaaagattacctacgatgttccagaacaagatttggaagttgacttgaactctattccaggttacgttaagtccggtttcgttattaagaacgttagagaagttgtcgacagaacttaa

SEQ ID NO: 3 - DNA, 8810 bp, Artificial Sequence
  Plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgagctccggatgcaagggttcgaatcccttagctctcattattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccataccatgggttggtctcacccacaattcgaaaagcatatggctactttgaagagagataagggtttggataacaccttgaaggttttgaagcaaggttacttgtacaccaccaatcaaagaaacagattgaacacctccgttttccaaacaaaagctttgggtggtaagccattcgttgttgttactggtaaagaaggtgctgaaatgttctacaacaatgacgttgttcaaagagaaggtatgttgccaaagagaattgtcaacactttgtttggtaagggtgccattcatactgttgatggtaagaaacacgttgacagaaaggctttgttcatgtctttgatgactgaaggtaacttgaactacgtcagagaattgactagaactttgtggcatgctaacacccaaagaatggaatctatggatgaagtcaacatctacagagaatccatcgttttgttgaccaaggttggtactagatgggctggtgttcaagctccaccagaagatattgaaagaattgctaccgatatggatattatgatcgactcctttagagctttaggtggtgcttttaaaggttacaaggcttccaaagaagccagaagaagagttgaagattggttggaagaacaaatcatcgaaaccagaaagggtaacattcatccacctgaaggtactgccttgtatgaatttgctcattgggaagattacttgggtaacccaatggattctagaacctgtgctattgatttgatgaacaccttcagaccattgatcgccattaacagatttgtttctttcggtttacacgccatgaacgaaaacccaattaccagagaaaagatcaagtccgaaccagattacgcttacaagtttgctcaagaagttagaagatattacccattcgtcccatttttgccaggtaaagctaaggttgatattgacttccaaggtgtcactattccagctggtgttggtttggctttggatgtttatggtactacccatgatgaatccttgtgggatgatccaaatgaattcagaccagaaagattcgaaacttgggatggttctccattcgatttgattccacaaggtggtggtgattactggactaatcatagatgtgccggtgaatggattaccgttattatcatggaagaaaccatgaagtactttgccgaaaagattacctacgatgttccagaacaagatttggaagttgacttgaactctattccaggttacgttaagtccggtttcgttattaagaacgttagagaagttgtcgacagaacttaaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 4 - DNA, 9139 bp, Artificial Sequence
  Plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaataaccatgggttggtctcacccacaattcgaaaagcatatggctactttgaagagagataagggtttggataacaccttgaaggttttgaagcaaggttacttgtacaccaccaatcaaagaaacagattgaacacctccgttttccaaacaaaagctttgggtggtaagccattcgttgttgttactggtaaagaaggtgctgaaatgttctacaacaatgacgttgttcaaagagaaggtatgttgccaaagagaattgtcaacactttgtttggtaagggtgccattcatactgttgatggtaagaaacacgttgacagaaaggctttgttcatgtctttgatgactgaaggtaacttgaactacgtcagagaattgactagaactttgtggcatgctaacacccaaagaatggaatctatggatgaagtcaacatctacagagaatccatcgttttgttgaccaaggttggtactagatgggctggtgttcaagctccaccagaagatattgaaagaattgctaccgatatggatattatgatcgactcctttagagctttaggtggtgcttttaaaggttacaaggcttccaaagaagccagaagaagagttgaagattggttggaagaacaaatcatcgaaaccagaaagggtaacattcatccacctgaaggtactgccttgtatgaatttgctcattgggaagattacttgggtaacccaatggattctagaacctgtgctattgatttgatgaacaccttcagaccattgatcgccattaacagatttgtttctttcggtttacacgccatgaacgaaaacccaattaccagagaaaagatcaagtccgaaccagattacgcttacaagtttgctcaagaagttagaagatattacccattcgtcccatttttgccaggtaaagctaaggttgatattgacttccaaggtgtcactattccagctggtgttggtttggctttggatgtttatggtactacccatgatgaatccttgtgggatgatccaaatgaattcagaccagaaagattcgaaacttgggatggttctccattcgatttgattccacaaggtggtggtgattactggactaatcatagatgtgccggtgaatggattaccgttattatcatggaagaaaccatgaagtactttgccgaaaagattacctacgatgttccagaacaagatttggaagttgacttgaactctattccaggttacgttaagtccggtttcgttattaagaacgttagagaagttgtcgacagaacttaaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 5 - DNA, 4241 bp, Artificial Sequence
  Plasmid construct
gcggccgcttttccaataggtggttagcaatcgtcttactttctaacttttcttaccttttacatttcagcaatatatatatatatatttcaaggatataccattctaccctcgaggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaataaccatgggttggtctcacccacaattcgaaaagcatatggctactttgaagagagataagggtttggataacaccttgaaggttttgaagcaaggttacttgtacaccaccaatcaaagaaacagattgaacacctccgttttccaaacaaaagctttgggtggtaagccattcgttgttgttactggtaaagaaggtgctgaaatgttctacaacaatgacgttgttcaaagagaaggtatgttgccaaagagaattgtcaacactttgtttggtaagggtgccattcatactgttgatggtaagaaacacgttgacagaaaggctttgttcatgtctttgatgactgaaggtaacttgaactacgtcagagaattgactagaactttgtggcatgctaacacccaaagaatggaatctatggatgaagtcaacatctacagagaatccatcgttttgttgaccaaggttggtactagatgggctggtgttcaagctccaccagaagatattgaaagaattgctaccgatatggatattatgatcgactcctttagagctttaggtggtgcttttaaaggttacaaggcttccaaagaagccagaagaagagttgaagattggttggaagaacaaatcatcgaaaccagaaagggtaacattcatccacctgaaggtactgccttgtatgaatttgctcattgggaagattacttgggtaacccaatggattctagaacctgtgctattgatttgatgaacaccttcagaccattgatcgccattaacagatttgtttctttcggtttacacgccatgaacgaaaacccaattaccagagaaaagatcaagtccgaaccagattacgcttacaagtttgctcaagaagttagaagatattacccattcgtcccatttttgccaggtaaagctaaggttgatattgacttccaaggtgtcactattccagctggtgttggtttggctttggatgtttatggtactacccatgatgaatccttgtgggatgatccaaatgaattcagaccagaaagattcgaaacttgggatggttctccattcgatttgattccacaaggtggtggtgattactggactaatcatagatgtgccggtgaatggattaccgttattatcatggaagaaaccatgaagtactttgccgaaaagattacctacgatgttccagaacaagatttggaagttgacttgaactctattccaggttacgttaagtccggtttcgttattaagaacgttagagaagttgtcgacagaacttaaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaactatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgattttatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgtaaaaagattctctttttttatgatatttgtacataaactttataaatgaaattcataatagaaacgacacgaaattacaaaatggaatatgttcatagggtaactagt

SEQ ID NO: 6 - DNA, 8120 bp, Artificial Sequence
  Plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaataaccatggtatgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 7 - DNA, 7944 bp, Artificial Sequence
  Plasmid construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaataaccatgggttggtctcacccacaattcgaaaagcatatgagtacgttggattcacattccctaaagttacagagcggctcgaagtttgtaaaaataaagccagtaaataacttgaggagtagttcatcagcagatttcgtgtccccaccaaattccaaattacaatctttaatctggcagaaccctttacaaaatgtttatataactaaaaaaccatggactccatccacaagagaagcgatggttgaattcataactcatttacatgagtcataccccgaggtgaacgtcattgttcaacccgatgtggcagaagaaatttcccaggatttcaaatctcctttggagaatgatcccaaccgacctcatatactttatactggtcctgaacaagatatcgtaaacagaacagacttattggtgacattgggaggtgatgggactattttacacggcgtatcaatgttcggaaatacgcaagttcctccggttttagcatttgctctgggcactctgggctttctatcaccgtttgattttaaggagcataaaaaggtctttcaggaagtaatcagctctagagccaaatgtttgcatagaacacggctagaatgtcatttgaaaaaaaaggatagcaactcatctattgtgacccatgctatgaatgacatattcttacataggggtaattcccctcatctcactaacctggacattttcattgatggggaatttttgacaagaacgacagcagatggtgttgcattggccactccaacgggttccacagcatattcattatcagcaggtggatctattgtttccccattagtccctgctattttaatgacaccaatttgtcctcgctctttgtcattccgaccactgattttgcctcattcatcccacattaggataaagataggttccaaattgaaccaaaaaccagtcaacagtgtggtaaaactttctgttgatggtattcctcaacaggatttagatgttggtgatgaaatttatgttataaatgaggtcggcactatatacatagatggtactcagcttccgacgacaagaaaaactgaaaatgactttaataattcaaaaaagcctaaaaggtcagggatttattgtgtcgccaagaccgagaatgactggattagaggaatcaatgaacttttaggattcaattctagctttaggctgaccaagagacagactgataatgattaaggatccgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttcgcagtttacactctcatcgtcgctctcatcatcgcttccgttgttgttttccttagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 8 - DNA, 20 bp, Artificial Sequence
  Amplification primer
tgggagaaaaaggaaaggtg

SEQ ID NO: 9 - DNA, 20 bp, Artificial Sequence
  Amplification primer
tgcaggtatgcgatagttcc

SEQ ID NO: 10 - DNA, 20 bp, Artificial Sequence
  Amplification primer
tcggatgcaaagttacatgg

SEQ ID NO: 11 - DNA, 20 bp, Artificial Sequence
  Amplification primer
tgccctcctccttgtcaata

SEQ ID NO: 12 - DNA, 20 bp, Artificial Sequence
  Amplification primer
actgtcgggcgtacacaaat

SEQ ID NO: 13 - DNA, 36 bp, Artificial Sequence
  Amplification primer
agccatggtatgccacattcctacgattacgatgcc

SEQ ID NO: 14 - DNA, 28 bp, Artificial Sequence
  Amplification primer
gtggatccttaaaacaggcggtttaaac

SEQ ID NO: 15 - DNA, 29 bp, Artificial Sequence
  Amplification primer
ctccatatgagtacgttggattcacattc

SEQ ID NO: 16 - DNA, 36 bp, Artificial Sequence
  Amplification primer
gaggatccttaatcattatcagtctgtctcttggtc

SEQ ID NO: 17 - DNA, 20 bp, Artificial Sequence
  Amplification primer
ctgcagcgaggagccgtaat

SEQ ID NO: 18 - DNA, 22 bp, Artificial Sequence
  Amplification primer
tgattttgatgacgagcgtaat

SEQ ID NO: 19 - DNA, 19 bp, Artificial Sequence
  Amplification primer
ggtgaaggacccatccagt

SEQ ID NO: 20 - DNA, 19 bp, Artificial Sequence
  Amplification primer
ggatggggttcaccctctg

SEQ ID NO: 21 - DNA, 20 bp, Artificial Sequence
  Amplification primer
atacggccttccttccagtt

SEQ ID NO: 22 - DNA, 19 bp, Artificial Sequence
  Amplification primer
gatttgcaggcatttgctc

SEQ ID NO: 23 - DNA, 25 bp, Artificial Sequence
  Amplification primer
cttcattcacgcacactactctcta

SEQ ID NO: 24 - DNA, 25 bp, Artificial Sequence
  Amplification primer
actaatggtagcttaactggcaatg

SEQ ID NO: 25 - DNA, 25 bp, Artificial Sequence
  Amplification primer
ggacataaaatacacaccgagattc

SEQ ID NO: 26 - DNA, 21 bp, Artificial Sequence
  Amplification primer
ccttgcaaccagcttcttctt

SEQ ID NO: 27 - DNA, 21 bp, Artificial Sequence
  Amplification primer
tcgtattcggatgatggacaa

SEQ ID NO: 28 - DNA, 25 bp, Artificial Sequence
  Amplification primer
tattcttttcctagtccaagtcctg

(19) United States
(12) Patent Application Publication
(10) Pub. No.: US 2014/0206057 A1
(43) Pub. Date: Jul. 24, 2014

(54) NOVEL YEAST STRAINS

(71) Applicant: Shell Oil Company, Houston, TX (US)

(72) Inventors: Annette ALCASABAS, Cambridge (GB); Paula Isabela DARLEY, Ware (GB); Pinar PIR, Cambridge (GB)

(73) Assignee: Shell Oil Company, Houston, TX (US)

(21) Appl. No.: 14/133,863

(22) Filed: Dec. 19, 2013

(30) Foreign Application Priority Data
Dec. 31, 2012 (EP) 12199839.7

(51) Int. Cl. C12P 5/02; C12N 15/81

(52) U.S. Cl. CPC C12P 5/026; C12N 15/815
USPC 435/167; 435/254.2; 435/254.22

(57) ABSTRACT

There is provided an alkene-producing yeast cell comprising a bacterial fatty acid decarboxylase enzyme, which may comprise the amino acid sequence SEQ ID NO:1 or a functional variant or portion thereof. The alkene may have 15, 17 or 19 carbon atoms. The cell may have a genome comprising a polynucleotide sequence encoding the bacterial fatty acid decarboxylase enzyme.

### CROSS-REFERENCE TO RELATED APPLICATIONS

This application claims priority to European Patent Application No. 12199839.7, filed on Dec. 31, 2012, the disclosure of which is incorporated herein by reference.

### TECHNICAL FIELD

The present disclosure relates to novel yeast strains useful for the production of alkenes, which are useful in the production of biofuels and/or biochemicals.

### BACKGROUND

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as admissions of any prior art.

With the diminishing supply of crude mineral oil, use of renewable energy sources is becoming increasingly important for the production of liquid fuels and/or chemicals. These fuels and/or chemicals from renewable energy sources are often referred to as biofuels. Biofuels and/or biochemicals derived from non-edible renewable energy sources are preferred as these do not compete with food production.

Alkenes are important constituents in the production of fuels and/or chemicals. It would therefore be desirable to produce alkenes (sometimes also referred to as bio-alkenes) from non-edible renewable energy sources.

U.S.2011/0196180 describes compositions and methods for producing bio-olefins. It describes the discovery of a novel gene, orf880, which encodes an olefin-producing enzyme. Polynucleotides described may be isolated from a bacterium, such as for example a bacterium of the genus Jeotgalicoccus. U.S.2011/0196180 describes a recombinant vector including the polynucleotide and a host cell comprising such a recombinant vector. Such host cell may express a polypeptide encoded by the recombinant vector. In U.S.2011/0196180 the heterologous expression of the orf880 gene of the bacterium Jeotgalicoccus ATCC 8456 in another bacterium, such as E. coli is exemplified. In passing, U.S.2011/0196180 also mentions a prophetic example (example 11), which suggests the production of alfa-olefins in Saccharomyces cerevisiae by heterologous expression, via a yeast expression vector, of Jeotgalicoccus sp. ATCC8456 orf880. Without wishing to be bound by any kind of theory, however, one skilled in the art may expect that it cannot successfully carry out this prophetic example. On the contrary, the method proposed in this example 11 for isolating alkenes, if they were indeed to be produced when using the prophetic example method, would be unlikely to be successful, because the YeastBuster™ extraction system suggested in the example makes use of detergent, which may be expected to bind to alkenes.

In addition to the use of a recombinant vector, U.S.2011/0196180 also mentions that the polynucleotide may be stably incorporated into the genomic DNA of the host cell. U.S.2011/0196180, however, provides no practical way of actually carrying out this wish.

It would be an advancement in the art to provide a yeast cell capable of producing alkenes in good yields. In addition, it would be an advancement in the art to provide a yeast cell capable of producing alkenes without making use of a heterologous expression vector.

### SUMMARY

A first aspect of the present disclosure provides an alkene-producing yeast cell comprising a bacterial fatty acid decarboxylase enzyme. The bacterial fatty acid decarboxylase enzyme may comprise the amino acid sequence SEQ ID NO:1 (the OleTje enzyme from the bacterium Jeotgalicoccus) or a functional variant or portion thereof.

In the bacterium Jeotgalicoccus, the last enzyme involved in alkene synthesis is a cytochrome P450 peroxygenase, OleTje, which decarboxylates fatty acid and produces carbon dioxide and a terminal alkene. Rude et al. (2011, Appl. Environ. Microbiol. vol. 77 p 1718-1727) have shown that this enzyme enables E. coli to produce alkenes from its own fatty acids.

The present inventors introduced recombinant OleTje into S. cerevisiae and successfully detected production of terminal alkenes by this organism. Alkenes ranging from C15 to C19 were identified in yeast cell lysates.

This is surprising, since not all genes that can be expressed in E. coli can be expressed as functional proteins in yeasts such as S. cerevisiae. Among potential problems are improper protein folding, lack of the necessary co-factors, difference in post-translational modification and protein degradation (reviewed by Romanos et al. (1992) Yeast vol. 8 p 423-488; Cereghino et al. (1999) Curr. Op. Biotechnol. vol. 10 p 422-427). These can lead to reduced protein production or inactive protein production. One example is the E. coli xylose isomerase gene which, although it can be expressed in S. cerevisiae, results in a protein which is inactive (Sarthy et al. (1987) Appl. Env. Microbiol. vol. 52 p 1996-2000). This is also true for the Bacillus xylose isomerase, which is functional in E. coli but not S. cerevisiae (Amore et al. (1989) Appl. Microbiol. Biotechnol. vol. 30 p 351-357).

The inventors expressed OleTje in S. cerevisiae by way of introducing a polynucleotide encoding the enzyme into the yeast genome. This is contrast to work proposed in prophetic Example 11 of U.S.2011/0196180, which suggested use of an expression vector carrying a polynucleotide encoding OleTje.

In one embodiment, there is provided an alkene-producing yeast cell comprising a bacterial fatty acid decarboxylase enzyme. In one embodiment, the bacterial fatty acid decarboxylase enzyme comprises the amino acid sequence SEQ ID NO:1 or a functional variant or portion thereof. In one embodiment, the cell comprises a polynucleotide encoding the bacterial fatty acid decarboxylase enzyme. In one embodiment, the polynucleotide comprises SEQ ID NO:2 or a functional variant or portion thereof. In one embodiment, the cell has a genome comprising the polynucleotide. In one embodiment, the cell is a Saccharomyces cell or an oleaginous yeast cell. In one embodiment, the cell is a member of the genus Yarrowia, Candida, Rhodotorula, Rhodosporidium, Cryptococcus, Trichosporon, or Lipomyces. In one embodiment, the cell is of the species Lipomyces starkeyi, Rhodosporidium toruloides, Rhodotorula glutinis, or Yarrowia lipolytica.

In one embodiment, the cell comprises at least one copy of a plasmid having nucleotide sequence SEQ ID NO:3 or 4 or a functional variant or portion thereof. In one embodiment, the alkene is a terminal alkene. In one embodiment, the alkene has 15, 16, 17, 18 or 19 carbon atoms. In one embodiment, the alkene has 15, 17 or 19 carbon atoms.

In one embodiment, the cell comprises a modification to provide an internal free fatty acid pool greater than the pool in a non-modified equivalent cell. In one embodiment, the cell comprises a disruption of an elo3 gene or a homologous gene thereof. In another embodiment, the cell comprises a disruption of a faa1 and a faa4 gene or a homologous gene thereof. In another embodiment, the cell comprises a modification to disrupt conversion of acetaldehyde to ethanol by the cell. In yet another embodiment, the cell comprises a disruption of an adh gene or a homologous gene thereof.

In one embodiment, the cell comprises a modification to provide an internal oxygen concentration greater than the concentration in a non-modified equivalent cell. In one embodiment, the cell comprises a disruption of a mitochondrial dehydrogenase-encoding gene.

In one embodiment, there is provided a method of preparing an alkene comprising culturing a cell as described herein. In one embodiment, the cell is cultured in a medium comprising at least one fatty acid. In one embodiment, the fatty acid comprises 16, 17, 18, 19 or 20 carbon atoms. In one embodiment, the method further comprises isolating an alkene from the cell by a method comprising a cell lysis method which does not include use of a detergent and a reducing agent. In one embodiment, the reducing agent is tris(hydroxypropyl)phosphine. In one embodiment, the method further comprises lysing the cell by agitation with glass beads.

In one embodiment, there is provided a method of producing an alkane, the method comprises hydrogenation of an isolated alkene produced by a cell described herein and/or in a method described herein. In one embodiment, there is provided a method of producing a branched alkane, the method comprises hydroisomerization of an isolated alkene produced by a cell a cell described herein and/or in a method described herein. In one embodiment, there is provided a method for the production of a biofuel and/or a biochemical comprising combining an alkene produced by a cell described herein and/or in a method described herein or an alkane produced in a method described herein with one or more additional components to produce a biofuel and/or biochemical. In one embodiment, there is provided use of a cell described herein as a biofuel/biochemical alkene precursor source.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and the benefit of this disclosure.

FIG. 1 shows lineage of strains constructed in this study from the parental FY2 strain.

FIG. 2 shows oleTje DNA constructs and expression in yeast, with A being a diagram of the chromosomal targeting construct used to introduce OleTje to yeast strain FY2 to create strain S368, B shows the OleTje overexpression construct pS362, a 2μ plasmid with the oleTje gene under the control of the MET25 promoter, C shows Western blot analysis of control strain YSBN8, single copy strain S368 and multiple copy strain S365.

FIG. 3A shows GC trace of oleTje strain S368 (lower trace) and control strain YSBN8 (upper trace), terminal alkenes 1-heptadecene (C17, 13.55 minutes) and 1-nonadecene (C19, 17 9 minutes) being marked with arrows, circles mark possible dienes, heptadecadiene (13.85 minutes) and nonadecadiene (18.1 minutes).

FIG. 3B shows mass spectrum of the peak at 17.9 min, identified as 1-nonadecene.

FIG. 3C shows mass spectrum of 2-methyl 5 octadecene standard, isomeric to 1-nonadecene.

FIG. 3D shows mass spectrum of the peak at 13 5 min from stearic acid-fed S368, identified as 1-heptadecene.

FIG. 3E shows mass spectrum of 1-heptadecene standard.

FIG. 4 shows the time course of alkene production in the single oleTje copy strain S368.

FIG. 5 shows that fatty acid feeding increases yield of heptadecene (C17) and nonadecene (C19) in the single oleTje copy strain S368.

FIG. 6 shows that overexpression of oleTje increases alkene yield.

FIG. 7 shows that NADPH-producing enzymes do not increase alkene yield in the multicopy oleTje strain S365 background.

FIG. 8 shows the pathway engineering in the oleTje yeast strains.

FIG. 9 shows pathway engineering in oleTje yeast strains increases alkene yield.

FIG. 10 shows that specific mutations increase alkene yield per biomass, in the single copy OleTje background, S368 (A) and combined with OleTje overexpression in the S365 background (B).

### DETAILED DESCRIPTION

Unless otherwise defined herein, scientific and technical terms used herein will have the meanings that are commonly understood by those of ordinary skill in the art.

Generally, nomenclatures used in connection with techniques of biochemistry, enzymology, molecular and cellular biology, microbiology, genetics, protein and nucleic acid chemistry and hybridization, described herein, are those well known and commonly used in the art.

Conventional methods and techniques mentioned herein are explained in more detail, for example, in Sambrook et al. (Molecular Cloning, a laboratory manual [second edition] Sambrook et al. Cold Spring Harbor Laboratory, 1989).

In this specification, SEQ ID NO:1 provides the amino acid sequence of the bacterial enzyme OleTje (Accession no. for the amino acid sequence is HQ709266) and SEQ ID NO:2 provides a S. cerevisiae codon-optimised DNA sequence encoding the OleTje protein, including a streptavidin tag-encoding sequence at the 5′ end (resides 1-33 of SEQ ID NO:2). The identity of other amino acid and nucleotide sequences referred to in this specification is as set out in Tables 2 and 3 at the end of the description. The terms “polynucleotide”, “polynucleotide sequence” and “nucleic acid sequence” are used interchangeably herein. The terms “polypeptide”, “polypeptide sequence” and “amino acid sequence” are, likewise, used interchangeably herein.

The cell according to the invention may be a recombinant cell. It may be a Saccharomyces cell or an oleaginous yeast cell. The oleaginous yeast cell may be a member of the genus Yarrowia, Candida, Rhodotorula, Rhodosporidium, Cryptococcus, Trichosporon, or Lipomyces and may be, by way of non-limiting example, a cell of the species Lipomyces starkeyi, Rhodosporidium toruloides, Rhodotorula glutinis, or Yarrowia lipolytica.

The cell may comprise a polynucleotide encoding the bacterial fatty acid decarboxylase enzyme. For example, the polynucleotide may comprise SEQ ID NO:2 or a functional variant or portion thereof. The polynucleotide may be included within (i.e., incorporated into) the genome of the cell. Therefore, the cell may have a genome comprising a polynucleotide encoding the bacterial fatty acid decarboxylase enzyme, such as a polynucleotide having sequence SEQ ID NO:2 or a functional variant or portion thereof. Advantages of preparing a cell according to the invention by means of inclusion in the cell genome of a polynucleotide encoding the bacterial fatty acid decarboxylase enzyme include the stable inheritance of the polynucleotide. This removes any need to carry out constant selection in order to maintain a vector. Furthermore, incorporation of a polynucleotide into the genome ensures a stable copy number, whereas vector copy number can fluctuate in cultures.

In particular embodiments, the cell may be transformed with and/or comprise at least one copy of a plasmid having nucleotide sequence SEQ ID NO:3 or 4 (pS362 and pS363, respectively), or a functional variant or portion of either of these.

The present invention also encompasses a cell comprising functional variants or portions of the bacterial fatty acid decarboxylase enzyme. As used herein, a “variant” means an enzyme in which the amino acid sequence differs from the base sequence from which it is derived in that one or more amino acids within the sequence are substituted for other amino acids, or deleted. For example, a variant of SEQ ID NO:1 may have an amino acid sequence at least about 60% identical to SEQ ID NO:1, for example, at least about 65%, 70%, 75%, 80%, 85%, 90%, 91%, 92%, 93%, 94%, 95%, 96%, 97%, 98%, 99% or about 100% identical. This may be the consequence of substitutions or deletions of amino acids within the original sequence SEQ ID NO:1. The variants or portions are functional variants or portions in that the variant sequence has similar or, preferably, identical functional fatty acid decarboxylase activity characteristics to the enzyme having the non-variant amino acid sequence (and this is the meaning of the term “functional variant or portion” as used throughout this specification). The similar or identical fatty acid decarboxylase characteristics as SEQ ID NO:1, mentioned above, may be assessed, for example, by comparing the rate of conversion of eicosanoic acid (C20) to nonadecene (C19) by a variant, to the rate achieved by SEQ ID NO:1 itself. For a functional variant, this rate may be the same or similar, for example at least about 60%, 70%, 80%, 90%, 95% or about 100% the rate achieved by SEQ ID NO:1. A functional variant may also have greater (i.e., an improved) activity than that of SEQ ID NO:1, i.e., the rate of conversion of eicosanoic acid to nonadecene may be more than 100% compared to that of SEQ ID NO:1.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H |

As is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

In the present invention, non-conservative substitutions are possible provided that these do not interrupt the enzyme activities of the polypeptides, as defined elsewhere herein.

Broadly speaking, fewer non-conservative substitutions than conservative substitutions will be possible without altering the biological activity of the polypeptides. Determination of the effect of any substitution (and, indeed, of any amino acid deletion or insertion) is wholly within the routine capabilities of the skilled person, who can readily determine whether a variant polypeptide retains the activity of the non-variant enzyme, as discussed above.

Using the standard genetic code, further nucleic acid sequences encoding the polypeptides may readily be conceived and manufactured by the skilled person, in addition to those disclosed herein. The nucleic acid sequence may be DNA or RNA and, where it is a DNA molecule, it may for example comprise a cDNA or genomic DNA. The nucleic acid may be contained within an expression vector, such as a plasmid as described elsewhere herein.

The invention, therefore, encompasses use of variant nucleic acid sequences encoding the polypeptides of the invention. The term “variant” in relation to a nucleic acid sequence means any substitution of, variation of, modification of, replacement of, deletion of, or addition of one or more nucleic acid(s) from or to a polynucleotide sequence, providing the resultant polypeptide sequence encoded by the polynucleotide exhibits at least the same or similar enzymatic properties as the polypeptide encoded by the basic sequence. The term therefore includes allelic variants and also includes a polynucleotide (a “probe sequence”) which substantially hybridises to the polynucleotide sequences disclosed herein. Such hybridisation may occur at or between low and high stringency conditions. In general terms, low stringency conditions can be defined as hybridisation in which the washing step takes place in a 0.330-0.825 M NaCl buffer solution at a temperature of about 40-48° C. below the calculated or actual melting temperature (Tₘ) of the probe sequence (for example, about ambient laboratory temperature to about 55° C.), while high stringency conditions involve a wash in a 0.0165-0.0330M NaCl buffer solution at a temperature of about 5-10° C. below the calculated or actual Tₘ of the probe sequence (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridisation of nucleic acid sequences have been described for example in Sambrook et al. (as above).

Typically, nucleic acid sequence variants have about 55% or more of the nucleotides in common with the nucleic acid sequence of the present invention, more typically about 60%, 65%, 70%, 80%, 85%, or even 90%, 95%, 98% or about 99% or greater sequence identity.

Variant nucleic acids may be codon-optimised for expression in a particular host cell.

Sequence identity between amino acid and nucleic acid sequences is determined herein by comparing an alignment of the sequences using the Needleman-Wunsch Global Sequence Alignment Tool available from the National Center for Biotechnology Information (NCBI), Bethesda, Md., USA, for example via http://blast.ncbi.nlm nih gov/Blast.cgi, using default parameter settings (for protein alignment, Gap costs Existence:11 Extension:1). Sequence comparisons and percentage identities mentioned in this specification have been determined using this software. When comparing the level of sequence identity to, for example, SEQ ID NO:1, this typically should be done relative to the whole length of SEQ ID NO:1 (i.e., a global alignment method is used). When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties. As mentioned above, the percentage sequence identity may be determined using the Needleman-Wunsch Global Sequence Alignment tool, using default parameter settings. The Needleman-Wunsch algorithm was published in J. Mol. Biol. (1970) vol. 48:443-53.

The cell according to the invention may produce an alkene which may be a terminal alkene and/or which may have 13, 14, 15, 16, 17, 18, 19, 20, 21, 22 or 23 carbon atoms, preferably 15, 17 or 19 carbon atoms. The alkene may be in linear or branched formation and comprise one or more double bonds. As is well known to the skilled person, an alkene is an unsaturated hydrocarbon comprising at least one carbon-to-carbon double bond. Particular examples of alkenes include straight- or branched-chain alkenes having an odd number of carbon atoms up to about 15, 17 or 19 carbon atoms. Particular examples of the alkenes have just a single inter-carbon double bond. In some embodiments, the cell produces a mixture of at least two alkenes each having 15, 16, 17, 18 or 19 carbon atoms. In a particular embodiment, where the cell produces a mixture of two or more alkenes, the cell produces an alkene having 19 carbon atoms and one or more further alkenes each having 15, 16, 17 or 18 carbon atoms.

For example, the cell may produce a mixture of alkenes comprising C17 at a concentration of at least 2 μg per litre of a liquid culture of the cell and C19 at a concentration of at least 5 μg per litre of the liquid culture, after about 2 days from the start of culture growth. In some embodiments, the cell may produce a mixture of alkenes comprising C17 at a concentration of at least about 1.5 μg, 1.6 μg, 1.7 μg, 1.8 μg, 1.9 μg, 2.0 μg, 2.5 μg, 3.0 μg, 3.5 μg, 4.0 μg, 4.5 μg, 5.0 μg, 5.5 μg, 6.0 μg, 6.1 μg, 6.2 μg, 6.3 μg, 6.4 μg or at least about 6.5 μg per litre of a liquid culture of the cell, after about 3 days from the start of culture growth. The mixture of alkenes may comprise C19 at a concentration of at least about 9 μg, 10 μg, 11 μg, 12 μg, 13 μg, 14 μg, 15 μg, 16 μg, 17 μg, or at least about 18 μg per litre of liquid culture, after about 3 days from the start of culture growth. In some embodiments, total alkene concentration may be at least about 30 μg, 31 μg, 32 μg, 33 μg, 34μ or at least about 35 μg per litre of liquid culture.

Total alkene yield may be at least about 0.5 μg/g dry cell mass, or at least about 1.3 μg/g, 1.4, 1.5, 1.6, 1.7, 1.8, 1.9, 2.0, 2.1, 2.2, 2.5, 3.0, 3.5, 4.0, 4.5, 5.0, 5.5, 6.0, 6.5, 7.0, 7.5, 8.0, 8.5, 9.0, 9.5, 10.0, 15.0, 20.0, 21.0, 22.0, 23.0, 24.0, or at least about 25.0 μg alkene per gram of dry cell mass.

The cell according to the invention may also comprise a modification to provide an internal free fatty acid pool greater than the pool in a non-modified equivalent cell. The term “modification” as used herein in relation to cells indicates that the cell has been altered in some way so as to alter the nucleic acid and/or polypeptide content of the cell in such a way as to cause a change in phenotype (in this case, the level of the internal free fatty acid pool). Such a modification may include, for example, a partial or whole deletion of a gene or other polynucleotide, which may result in the presence, absence or modification of a polypeptide sequence. A “non-modified equivalent cell” is a cell which is identical to the modified cell apart from the modification concerned.

Reference to “internal free fatty acid pool” indicates the concentration of free fatty acids within a cell, which may be determined using a gas chromatography method such as is described herein. An alternative method is as described in Sakuradani et al. (1999, Eur. J. Biochem. vol. 261 p 812-820). The fatty acid or acids contained in the fatty acid pool may be selected from fatty acids comprising 16, 17, 18, 19 or 20 carbon atoms. In the absence of additional modifications, fatty acids found in yeast cells typically have an even number of carbon atoms.

By way of example, a cell comprising a modification to provide an internal free fatty acid pool may comprise a disruption of an elo3 gene or a homologous gene thereof. The gene elo3 is the S. cerevisiae gene (NCBI Gene ID851087) which encodes a long chain acyl elongase. Disruption of this gene increases the pool of fatty acids having, for example, 20 carbon atoms. Homologous genes are found in other yeasts and can be readily identified by the skilled person. (For example, suitable genes may include putative elongation of fatty acids protein ELOH1 from Schizosaccharomyces pombe, UniProt ID Q9UTF7; unreviewed protein with UniProt ID Q6CDY7 from Yarrowia lipolytica; UniProt C5M5P9 from Candida tropicalis; UniProt Q6FT73 from Candida glabrata, uncharacterized fatty acid elongases from Pichia spp. with UniProt IDs E7RBX9, Q50L63, C4R3Y7, F2QVY3) A cell comprising such a modification may produce total alkene at least about 116 μg/litre of liquid cell culture after about 3 days from the start of culture growth, equivalent to 23 μg alkene per gram of dry cell weight. This may represent about a 10-, 20-, 30-, 40- or about a 50-fold increase in comparison to the total alkene production of a non-modified equivalent cell.

“Disruption” of a gene, as referred to throughout this specification, indicates that the activity of the gene is reduced in some way. For example, the gene may be wholly or partially absent from the cell, as compared to an equivalent cell which does not comprise the modification, or the sequence of the gene may be altered so as to reduce activity (for example, the level of transcription achieved from the gene by the cell). Therefore, this encompasses deletion of, substitution of and addition of nucleotides within the gene.

Alternatively or additionally to the disruption of an elo3 gene or homologous gene, a cell comprising a modification to provide an internal free fatty acid pool may comprise a disruption of both a faa1 and a faa4 gene or of the homologous genes thereof. The genes faa1 and faa4 are S. cerevisiae genes encoding acyl-CoA synthetases. The S. cerevisiae gene fat1 (UniProt ID P38225), also encoding an acyl-CoA synthetase, may also be suitable. Homologous genes are found in other yeasts and can be readily identified by the skilled person. (For example, suitable genes may include Long-chain-fatty-acid-CoA ligase 1 (UniProt ID C5MID6) from Candida tropicalis, unreviewed protein (UniProt ID Q6C8Q3 or YALIOD17864p) from Yarrowia lipolytica, LCF1 (UniProt ID 060135) and LCF2 (UniProt ID Q9P7D7) from Schizosaccharomyces pombe, long chain fatty acyl-CoA synthetase (UniProt ID C4R1R9) from Komagataella pastoris, long chain fatty acyl-CoA synthetase(UniProt ID E7RA39) from Pichia augusta.) A cell comprising such a modification may produce total alkene at 96 μg/litre of liquid cell culture after about 3 days, equivalent to 6 μg alkene per gram of dry cell weight. This may represent about a 3-, 4-, 5-, 6-, 7-, 8-, 9-, 10-, 11-, 12-, 13-, 14- or about a 15-fold increase in comparison to the total alkene production of a non-modified equivalent cell.

Alternatively or additionally, a cell according to the invention may comprise a modification to disrupt conversion of acetaldehyde to ethanol by the cell. “Disrupt conversion” indicates that the pathway of conversion of acetaldehyde to ethanol in the cell is disrupted so that a reduced amount or no ethanol is produced from acetaldehyde by the cell, in comparison to the amount produced in the absence of the modification. For example, the cell may comprise a disruption of an adh gene or a homologous gene thereof. The gene adh1 is a S. cerevisiae gene encoding an alcohol dehydrogenase. Homologous genes are found in other yeasts and can be readily identified by the skilled person. A cell comprising such a modification may produce total alkene at at least about 1.1 μg, 1.2 μg, 1.3 μg, 1.4 μg or at least about 1.5 μg/g dry cell weight after about 3 days from the start of culture growth

Alternatively or additionally, a cell according to the invention may comprise a modification to provide a predicted internal oxygen concentration greater than the predicted concentration in a non-modified equivalent cell. The internal oxygen concentration may be determined through metabolic modelling, specifically flux balance analysis (FBA) (see, for example, U.S. Pat. No. 7,751,981). For example, the cell may comprise a disruption of a mitochondrial dehydrogenase-encoding gene such as the S. cerevisiae gene mtd1 or homologues from other yeasts.

Particular examples of cells according to embodiments of the invention include strains having the genotypes of S364, S365, S366, S367, S368, S431, S432, and S437 as set out in Table 1 below, especially S431 (genotype MATα ura3-52 leu2Δ::PGKp-oleTje::hph elo3Δ::kanMX4+plasmid S362 2μ(LEU2, MET25p-oleTje)) and S437 (genotype MATα ura3-52 leu2Δ::PGKp-oleTj e::hph faa1Δ::kanMX4 faa4Δ::natMX +plasmid S362 2μ(LEU2, MET25p-oleTje)).

According to a second aspect of the invention, there is provided a method of preparing an alkene comprising culturing a cell according to the first aspect of the invention. The term “culturing a cell” indicates growing a cell or a sample of cells in liquid media or on a solid medium such as an agar plate. This may be done under controlled conditions such as at a fixed temperature (30° C. is typical) and/or fixed oxygen concentration, for example. The contents of the solid or liquid media may be altered by the user to affect the growth or other aspects of the phenotype of the cells. Such culturing methods are extremely well understood by the skilled person and particular details of suitable methods are also referred to in the Examples below.

The method according to the second aspect of the invention may subsequently comprise isolating the alkene and/or mixture of alkenes. The term “isolating the alkene” indicates that the alkene, or mixture of alkenes, is separated from other non-hydrocarbon components. This may indicate that, for example, at least about 50% by weight of a sample after separation is composed of the alkene(s), for example, at least about 60%, 70%, 75%, 80%, 85%, 90%, 95%, 96%, 97%, 98% 99% or 100%. The alkene produced during the working of the invention can be separated (i.e., isolated) by any known technique. One exemplary process is a two-phase (bi-phasic) separation process, involving conducting the method for a period and/or under conditions sufficient to allow the alkene(s) to collect in an organic phase and separating the organic phase from an aqueous phase. This may be especially relevant in the context of alkene production by a yeast cell, as is the case in the present invention. Bi-phasic separation uses the relative immiscibility of hydrocarbons to facilitate separation “Immiscible” refers to the relative inability of a compound to dissolve in water and is defined by the compound's partition coefficient, as will be well understood by the skilled person.

In an embodiment, the further step of isolating the alkene may comprise a step of lysing the cell or cells, which is preferably achieved by a method which does not involve use of a detergent and/or a tris(hydroxypropyl)phosphine (THP) reducing agent. Cell lysis may be achieved by mixing the cell or cells with beads comprising a hard material such as glass and subjecting the mixture to agitation.

In the method according to the second aspect of the invention, the cell or cells may be cultured in a medium comprising at least one fatty acid, for example a fatty acid comprising 16, 17, 18, 19 or 20 carbon atoms. This assists with maximising the internal free fatty acid pool in the cell, so that the concentration of the fatty acid substrate is not the rate limiting factor on enzyme activity. Culturing (or “feeding”) with a fatty acid having an odd number of carbon atoms, not naturally present in yeast, will enable production of an alkene having an even number of carbon atoms. Suitable methods are disclosed, for example, in Dittrich et al. (1998; Eur. J. Biochem. vol. 25 p 477-485).

According to a third aspect of the invention, there is provided a method of producing an alkane, comprising hydrogenation of an isolated alkene produced by a cell according to the first aspect of the invention and/or in a method according to the second aspect of the invention.

The unsaturated bonds in the isolated alkene can be hydrogenated to produce the alkane. Hydrogenation may be carried out in any manner known by the person skilled in the art to be suitable for hydrogenation of unsaturated compounds. The hydrogenation catalyst can be any type of hydrogenation catalyst known by the person skilled in the art to be suitable for this purpose. The hydrogenation catalyst may comprise one or more hydrogenation metal(s), for example, supported on a catalyst support. The one or more hydrogenation metal(s) may be chosen from Group VIII and/or Group VIB of the Periodic Table of Elements. The hydrogenation metal may be present in many forms; for example, it may be present as a mixture, alloy or organometallic compound. The one or more hydrogenation metal(s) may be chosen from the group consisting of Nickel (Ni), Molybdenum (Mo), Tungsten (W), Cobalt (Co) and mixtures thereof. The catalyst support may comprise a refractory oxide or mixtures thereof, for example, alumina, amorphous silica-alumina, titania, silica, ceria, zirconia; or it may comprise an inert component such as carbon or silicon carbide.

The temperature for hydrogenation may range from, for example, 300° C. to 450° C., for example, from 300° C. to 350° C. The pressure may range from, for example, 50 bar absolute to 100 bar absolute, for example, 60 bar absolute to 80 bar absolute.

A fourth aspect of the invention provides a method of producing a branched alkane, comprising hydroisomerization of an isolated alkene produced in by a cell according to the first aspect of the invention and/or a method according to a second aspect of the invention, or an alkane produced in a method according to the third aspect of the invention. Hydroisomerization may be carried out in any manner known by the person skilled in the art to be suitable for hydroisomerization of alkanes. The hydroisomerization catalyst can be any type of hydroisomerization catalyst known by the person skilled in the art to be suitable for this purpose. The one or more hydrogenation metal(s) may be chosen from Group VIII and/or Group VIB of the Periodic Table of Elements. The hydrogenation metal may be present in many forms, for example it may be present as a mixture, alloy or organometallic compound. The one or more hydrogenation metal(s) may be chosen from the group consisting of Nickel (Ni), Molybdenum (Mo), Tungsten (W), Cobalt (Co) and mixtures thereof. The catalyst support may comprise a refractory oxide, a zeolite, or mixtures thereof. Examples of catalyst supports include alumina, amorphous silica-alumina, titania, silica, ceria, zirconia; and zeolite Y, zeolite beta, ZSM-5, ZSM-12, ZSM-22, ZSM-23, ZSM-48, SAPO-11, SAPO-41, and ferrierite.

Hydroisomerization may be carried out at a temperature in the range of, for example, from 280 to 450° C. and a total pressure in the range of, for example, from 20 to 160 bar (absolute).

In one embodiment, hydrogenation and hydroisomerization are carried out simultaneously.

A fifth aspect of the invention provides a method for the production of a biofuel and/or a biochemical comprising combining an alkene produced by a cell according to the first aspect of the invention and/or in a method according to the second aspect of the invention with one or more additional components to produce a biofuel and/or biochemical.

According to a sixth aspect of the invention, there is provided a method for the production of a biofuel and/or a biochemical comprising combining alkane produced according to the third or fourth aspects with one or more additional components to produce a biofuel and/or biochemical.

In the fifth and sixth aspects, the alkane and/or alkene can be blended as a biofuel component and/or a biochemical component with one or more other components to produce a biofuel and/or a biochemical. By a biofuel or a biochemical, respectively, is herein understood a fuel or a chemical that is at least partly derived from a renewable energy source. Examples of one or more other components with which the alkane and/or alkene may be blended include anti-oxidants, corrosion inhibitors, ashless detergents, dehazers, dyes, lubricity improvers and/or mineral fuel components, but also conventional petroleum derived gasoline, diesel and/or kerosene fractions.

A further aspect of the invention provides the use of a cell according to the first aspect of the invention as a biofuel/biochemical alkene precursor source. A “biofuel/biochemical alkene precursor” is an alkene or mixture of alkenes, which may be used in the preparation of a biofuel and/or a biochemical, for example in a method according to the fifth or sixth aspects of the invention. The use of a cell as the source of such a precursor indicates that the cell according to the first aspect of the invention produces alkenes suitable for use in the biofuel/biochemical production methods, the alkenes being isolatable from the cell as described elsewhere herein.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

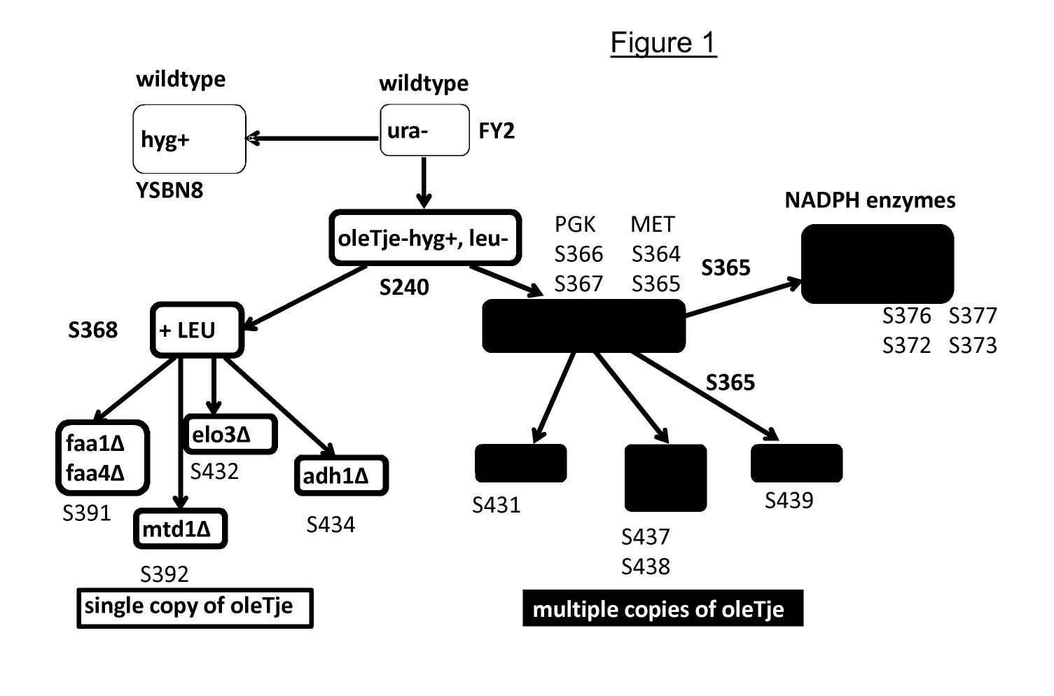

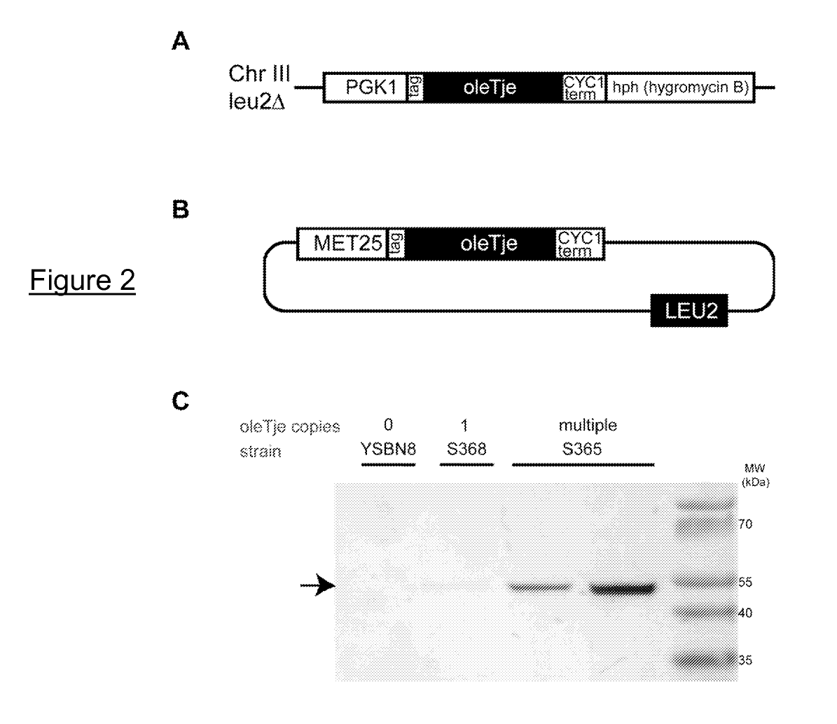

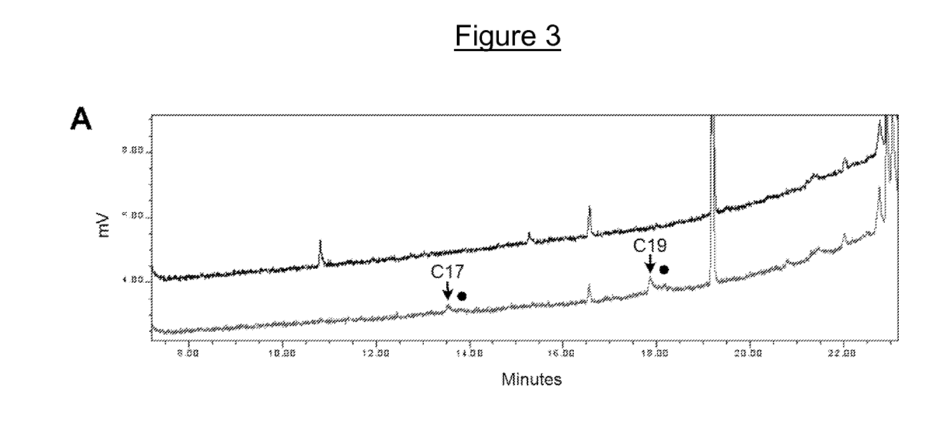

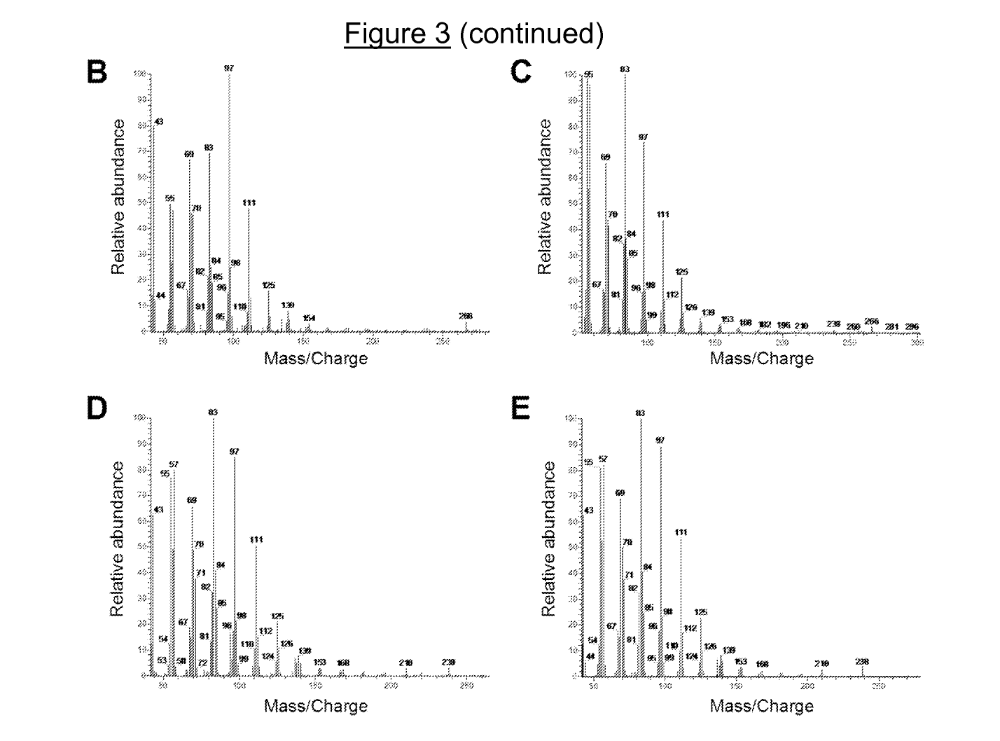

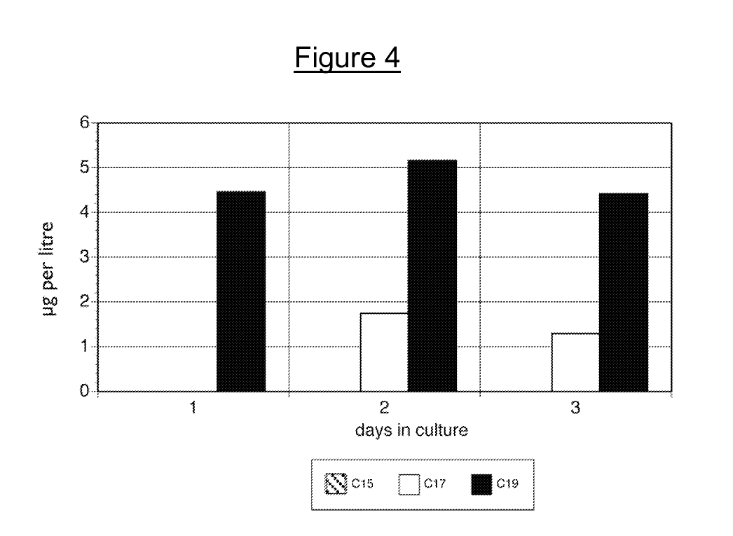

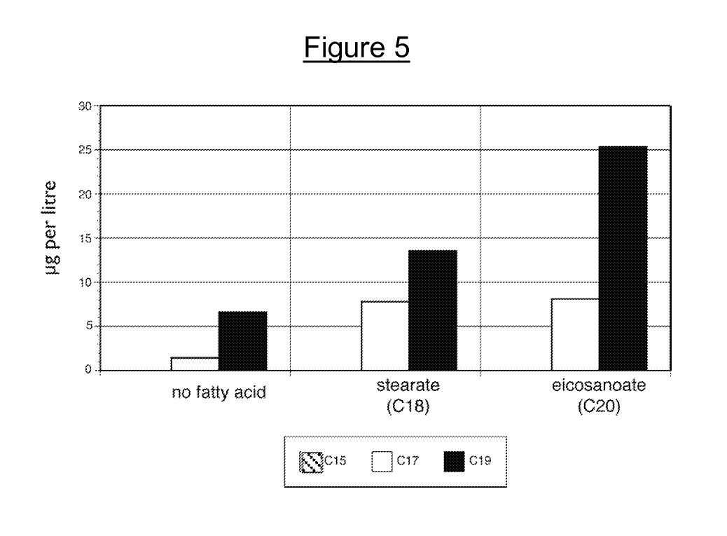

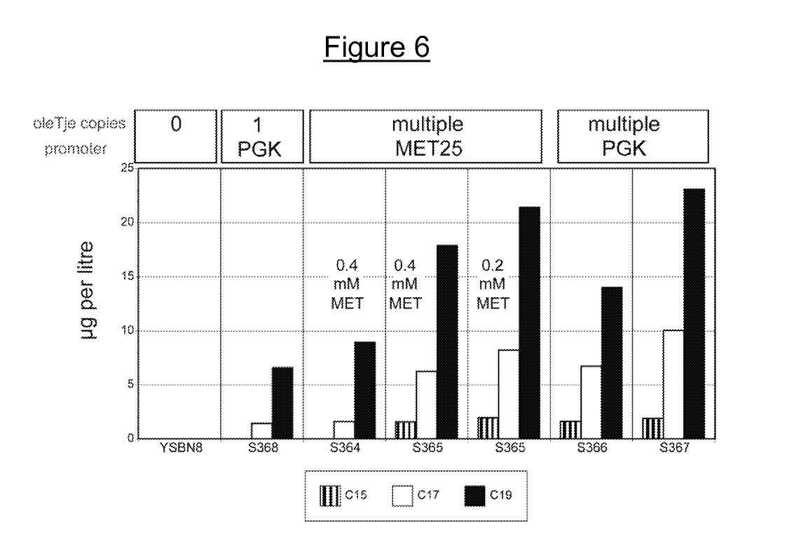

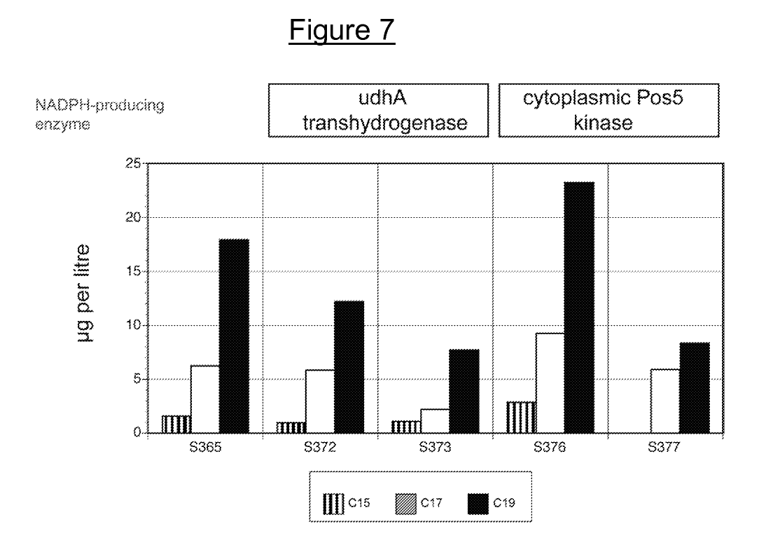

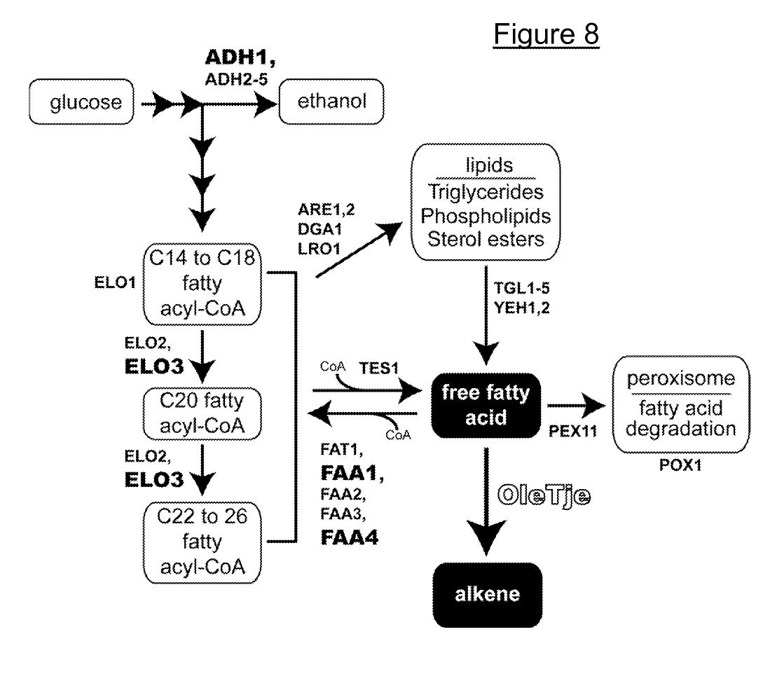

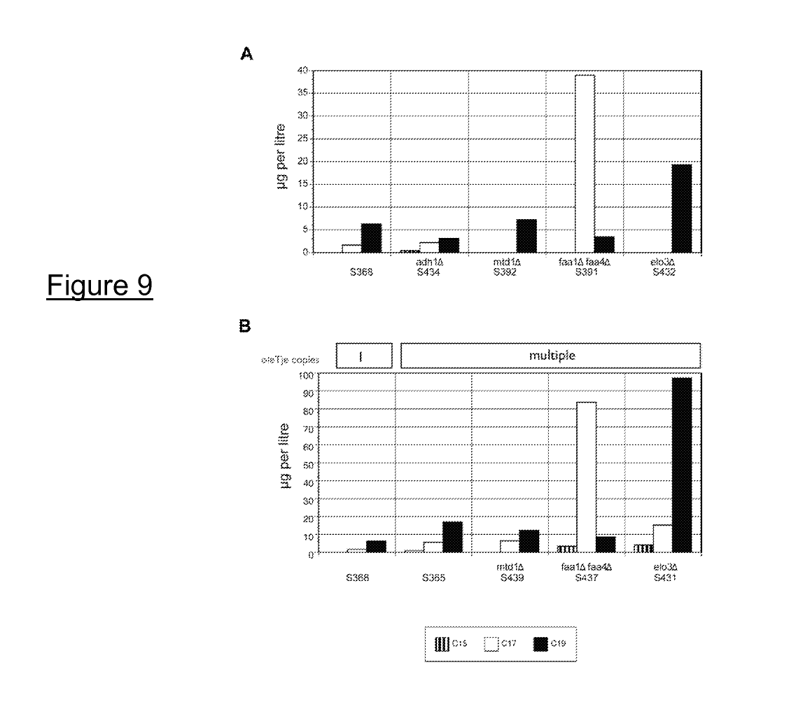

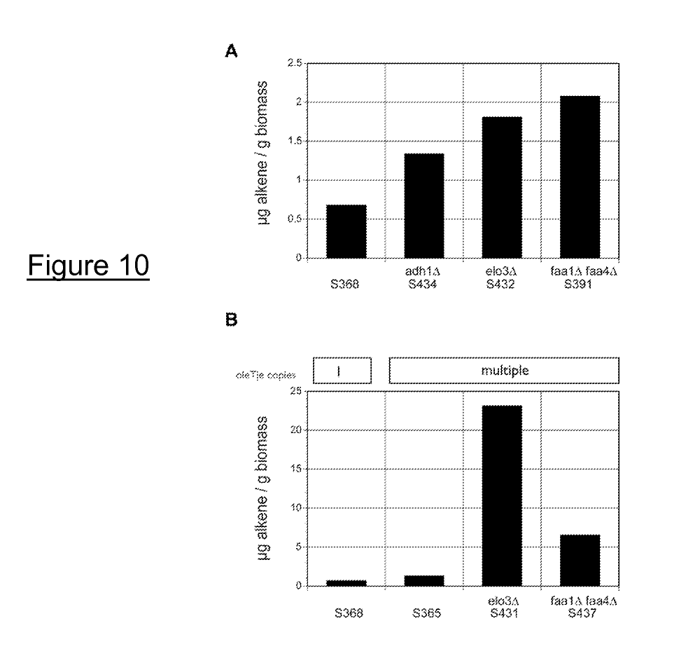

Embodiments of the invention will now be described, by way of example only, with reference to the following FIGS. 1-10. FIG. 1 shows lineage of strains constructed in this study from the parental FY2 strain. FIG. 2 shows oleTje DNA constructs and expression in yeast, with A being a diagram of the chromosomal targeting construct used to introduce OleTje to yeast strain FY2 to create strain 5368, B shows the OleTje overexpression construct pS362, a 2μ plasmid with the oleTje gene under the control of the MET25 promoter, C shows Western blot analysis of control strain YSBN8, single copy strain S368 and multiple copy strain 5365. FIG. 3A shows GC trace of oleTje strain S368 (lower trace) and control strain YSBN8 (upper trace), terminal alkenes 1-heptadecene (C17, 13.55 minutes) and 1-nonadecene (C19, 17.9 minutes) being marked with arrows, circles mark possible dienes, heptadecadiene (13.85 minutes) and nonadecadiene (18.1 minutes). FIG. 3B shows mass spectrum of the peak at 17.9 min, identified as 1-nonadecene. FIG. 3C shows mass spectrum of 2-methyl 5 octadecene standard, isomeric to 1-nonadecene. FIG. 3D shows mass spectrum of the peak at 13.5 min from stearic acid-fed S368, identified as 1-heptadecene. FIG. 3E shows mass spectrum of 1-heptadecene standard. FIG. 4 shows the time course of alkene production in the single oleTje copy strain S368. FIG. 5 shows that fatty acid feeding increases yield of heptadecene (C17) and nonadecene (C19) in the single oleTje copy strain S368. FIG. 6 shows that overexpression of oleTje increases alkene yield. FIG. 7 shows that NADPH-producing enzymes do not increase alkene yield in the multicopy oleTje strain S365 background. FIG. 8 shows the pathway engineering in the oleTje yeast strains. FIG. 9 shows pathway engineering in oleTje yeast strains increases alkene yield. FIG. 10 shows that specific mutations increase alkene yield per biomass, in the single copy OleTje background, S368 (A) and combined with OleTje overexpression in the S365 background (B).

### EXAMPLES

### Materials And Methods

### Yeast Strain Construction

Yeast strains (Table 1 below and FIG. 1) were all constructed from strain FY2 (Winston et al. (1995) Yeast vol. 11 p 53-55). The oleTje gene (GenBank HQ709266; Rude et al., 2011) was codon-optimised for S. cerevisiae and synthesized by GeneArt (Invitrogen).

To introduce the oleTje gene and delete native yeast genes, the inventors used a high-efficiency lithium acetate transformation protocol (Gietz et al. (2007) Nature Protocols vol. 2 p 35-37). Transformed strains (FY2, S368, or S365) were plated out on YPD-agar containing 300 μg/ml hygromicin B (Calbiochem) to select for the hph gene, 200 μg/ml G418 (Sigma) to select for the kan gene, or 100μg/ml clonNAT (Werner Bioagents) to select for the nat1 gene. The oleTje integration construct (FIG. 2A) and high-copy plasmid (FIG. 2B) are described in the Results section below.

To introduce NADPH-producing enzymes into yeast, the genes udhA and truncated POS5 were amplified using genomic DNA from E. coli strain JM109 and S. cerevisiae strain FY2, respectively, using primers listed in Table 3. They were cloned between the PGK1 promoter and CYC1 terminator into yeast vector pRS426 (Sikorski and Hieter (1989) Genetics vol. 122 p 19-27) and transformed into oleTje yeast strain S365. Transformants were selected on synthetic complete medium without leucine and uracil.

To delete specific genes in yeast strains harbouring oleTje, deletion constructs of the genes MTD1, ADH1, ELO3 and FAA1 were amplified from genomic DNA of the appropriate strains available through the yeast deletion collection (Open Biosystems; Winzeler et al. (1999) Science vol. 285 p 901-906). PCR products were directly transformed into both S368 and S365, and transformants selected for the kan gene. For FAA4, the faa4 deletant was first converted to carry the nat1 marker (as described by Tong and Boone (2006) Methods in Molecular Biology (Clifton, N.J.), vol. 313 p 171-192). The deletion cassette was then amplified and the PCR product transformed into the oleTje faa1 strains. All deletions were confirmed by colony PCR. All primers are listed in Table 3.

### Expression of OleTje In Oleaginous Yeasts

The expression of oleTje in an oleaginous yeast, such as Yarrowia lipolytica, can be achieved by transformation of a plasmid bearing oleTje under control of the strong promoter Hp4d using the lithium acetate procedure (molecular tools for Yarrowia reviewed by Nicaud (2012) Yeast, 29 (10, 409-418). Alkene production can be tested using the protocols the inventors developed for Saccharomyces cerevisiae. Genetic modifications of the Yarrowia homologues of ELO3 and the FAA genes could also be tested for increased alkene production.

### Yeast Media And Culture Conditions

Yeast strains were grown in synthetic complete (SC) medium without leucine and containing 2% glucose. S365-based strains with oleTje expression driven by the MET25 promoter were grown under either conditions of transcriptional derepression (0.2 mM methionine) or repression (2 mM methionine). In fatty-acid feeding experiments, synthetic complete medium was supplemented with 0.02% fatty acid and 1% tergitol (similar to the media used by Lockshon et al. (2007) Genetics vol. 175 p 77-91). All cultures were grown at 30° C. Liquid cultures (50 to 60 ml) were grown from a starting OD600 of 0.01, in 250 ml flasks at 210 rpm shaking. Cells were collected for alkene extraction after 3 days, with the exception of the time course experiment where collection times are stated.

### Heterologous Protein Production And Western Blot Analysis

In all the DNA constructs, the codon-optimised oleTje gene was fused to an N-terminal streptavidin tag sequence. Total cell protein was extracted from 1 ml of a 24-hour culture, using the trichloroacetic acid precipitation method (Paciotti et al., 1998); protein concentration was measured using the Bradford assay. An aliquot (25 μg) of total protein extract was run on a 10-20% polyacrylamide gel with Tricine buffer (Invitrogen) and transferred onto a PVDF membrane. This was incubated for 1 hour with 1:4000 dilution of Strep-Tactin alkaline phosphatase conjugate (IBA). After washing the membrane, it was incubated in BCIP/NBT substrate solution (Sigma) until distinct bands corresponding to the tagged proteins were visible (10-20 minutes).

### Extraction And Quantification of Alkenes

Alkenes, fatty acid ethyl esters and other hydrophobic metabolites were extracted from yeast cell lysates. Cell pellets from 50 or 60 ml cultures were collected and mixed in glass vials with 100 μl water and 200 μl of acid-washed 0.5 mm glass beads (Biospec). Cells were then lysed by shaking at 5.5 m/s for a total of 3 minutes in a FastPrep (MPBio). Internal standards (5 μg each of tetradecane, tetradecene, ethyl heptanoate, and pentadecanol) and 0.5 ml hexane were added to each vial. Solutions were mixed for 1 hour at 2000 rpm and subsequently frozen for 10 minutes to 2 hours at −80° C. Centrifugation of frozen samples (3600 rpm, 4° C., 25 minutes) was done to separate and collect the hexane layer.

Hexane extracts were run in a Gas Chromatograph (Perkin Elmer) using a Zebron FFAP column (30 m×0.25 mm×0.25 μm, Phenomenex) with helium at a flow rate of 2.4 ml/min. The temperature profile was as follows: The initial oven temperature was 70° C., it was then ramped up at a rate of 5° C./min to 160° C. In order to obtain a good separation for all standards, a second temperature ramp was needed at a rate of 8° C./min to 250° C. and held at 250° C. for 7 min. Alkenes and other metabolites were identified and quantified against known standards (Table 4). To confirm the identification of alkenes and ethyl esters, selected samples and controls were also run on a GC-MS (Thermofisher DSQII) using the same Zebron FFAP column and temperature profile, flow rate of 1.5ml/min helium, 1 μl cold on-column injection, electron impact ionization, and a scan rate of 45-645 daltons at 0.5 sec/scan. For identification, the mass spectra of selected peaks were compared to a reference library (Stein (retrieved 2012) “Mass Spectra” in NIST Chemistry WebBook, NIST Standard Reference Database Number 69, http://webbook.nist.gov) as well as the mass spectra of known standards.

### Flux Balance Analysis

The S. cerevisiae genome-wide metabolic model iIN800 (Nookaew et al. (2008) BMC Syst. Biol. 2:71; doi: 10.1186/1752-0509-2-71) was downloaded from http://sourceforge.net/projects/yeast/files/other/. The MATLAB/COBRA platform was used to carry out Flux Balance Analysis (FBA). The model was modified for (1) addition of oleTje and (2) gene deletions for relevant auxotrophies. Simultaneous biomass and alkene production was simulated for all single-deletion mutants. Deletion mutants identified by the simulations as increasing alkene production without any major impact on biomass production were selected and tested in vivo. The model was further extended to represent the production of by-products (ethyl esters) found in GC-MS analysis of the cell extracts and further predictions were made.

### EXAMPLES

### Results

### Heterologous oleTje Expression In Yeast

To test the activity of the OleTje protein in yeast, a single copy of the codon-optimised oleTje gene was integrated into the genome of S. cerevisiae strain FY2 (Winston et al. (1995) Yeast vol. 11 p 53-55). FY2 is derived from S288C, a strain whose complete genome sequence is available (http://www.yeastgenome.org) and this facilitated the design of the constructs for genetic manipulation of the production strain. OleTje was fused to the streptavidin tag and placed under control of the constitutive PGK1 promoter and CYC1 terminator, the genomic integration site was the LEU2 gene on chromosome III (FIG. 2A).

The resulting strain, 5240 (Table 1), has a complete deletion of the LEU2 gene, resulting in leucine auxotrophy. The inventors took advantage of this phenotype to overexpress oleTje by introducing high copy-number 2μ plasmids bearing both the wild-type LEU2 gene and oleTje into 5240; these plasmids are stably maintained in yeast grown without leucine. 2μ plasmids were also constructed where oleTje is under control of the regulatable promoter MET25 (pS362, FIG. 2B and Table 2) and PGK1 (pS363, Table 2). Strain 5365 contains the pS362, and oleTje expression is induced at methionine concentrations below 0.5 mM (Mumberg et al., 1994). Strain 5368 was also constructed, which contains an integrated copy of oleTje and plasmid pRS425 (Sikorski and Hieter (1989) Genetics vol. 122 p 19-27) to restore leucine prototrophy.

Both the S368 (single-copy) and S365 (multicopy) strains produced the streptavidin-tagged OleTje protein, visible as a 50 kDa band on the Western blot shown in FIG. 2C; the same size as the recombinant polyhistidine-tagged OleTje produced by Rude and colleagues (2011; Appl. Eviron. Microbiol. vol. 77 p 1718-1727). Strain S368, which only has the single copy of oleTje, produced a small amount of protein, faintly visible on the Western blot under these conditions. S365, bearing multiple copies of the oleTje gene, produced a higher concentration of the protein than S368, even under repressed conditions (2 mM methionine) and protein production was further increased under derepressed conditions (no methionine). YSBN8, a hygromycin-resistant version of FY2, was used as a negative control strain (Canelas et al. (2010) Nature Communs. 1:-; doi:10.1038/ncomms 1150). All strains constructed in this study are listed in Table 1 and their lineage from the original FY2 strain is outlined in FIG. 1.

### Alkene Production In the Single Copy oleTje Strain

Initial experiments were carried out on S368 to determine whether a single copy of the oleTje gene, expressed from a strong constitutive promoter, would be sufficient to produce alkenes in yeast. S368 was grown in SC medium without leucine and with 2% glucose for three days. Using GC analysis on yeast cell extracts, the alkene 1-nonadecene (C19, retention time 17.9 minutes) was observed as the major product, followed by 1-heptadecene (C17, 13.5 minutes); see FIG. 3A. These products were identified based on the retention times of known standards (Table 4) and were subsequently confirmed using GC-MS (FIGS. 3B, 3C, 3D, 3E). The mass spectrum of the peak corresponding to 1-nonadecene (FIG. 3B) is consistent with that of a 19-carbon alkene based on reference spectra from the National Institute of Science and Technology mass spectra database (Stein (retrieved 2012) “Mass Spectra” in NIST Chemistry WebBook, NIST Standard Reference Database Number 69, http://webbook.nist.gov) and is similar to the isomeric standard 2-methyl 5 octadecene (FIG. 3C). There was not enough of the 1-heptadecene peak (C17, 13 5 minutes in FIG. 2A) to be identified using GC-MS; however stearic acid-fed 5368 (described below) produced a higher amount of this peak and its mass spectra (FIG. 3D) is identical to that of the 1-heptadecene standard (FIG. 3E).

Next, alkene production was quantitated in S368 over the course of three days (FIG. 4). Nonadecene was produced after 1 day, that is, during early stationary phase. Its concentration increased after 2 days to over 5 μg per litre of culture. Heptadecene was observed from 2 days at under 2 μg/litre of culture. The highest level of both alkenes was measured within the cells at 2 days, this dropped by 17% on the third day. This suggests that alkenes are either consumed or secreted by yeast after the second day. The latter was tested by examining alkenes extracted from 1.5 ml samples of medium for all three days; however, they were either absent or their concentration was too low to be detectable using the available extraction and GC protocol (data not shown). Total alkenes produced adjusted for dry cell weight was 0.6 μg/g.

In yeast, the major fatty acids are C16 and C18, comprising 97% (20% for the saturated forms, 76.6% for the monounsaturated forms) of the free fatty acid pool, whereas C20 represents 0.23% (Oh et al. (1997) J. Bio. Chem. vol. 272 p 17376-17384). It was therefore unexpected to have nonadecene, a product of the saturated C20 fatty acid, as the major alkene product. This suggests a much greater preference of the OleTje enzyme for the C20 fatty acid. In the Rude et al. study mentioned above, OleTje enzyme decarboxylated both C18 and C16 fatty acids in E. coli; however, in Jeotgalicoccus, branched and unbranched forms of the C20 fatty acid are the enzyme's main substrates.

The mono-unsaturated forms are the most abundant fatty acid species in yeast (typically 80% of total free fatty acids, depending on growth conditions; reviewed in Tehlivets et al. (2007) Biochim et Biophys. Acta. vol. 1771 p 255-270). It was therefore surprising not to find dienes as the major products, since they would be formed by the decarboxylation of abundant fatty acids such as palmitoleic acid (C16) and oleic acid (C18). There were, however, smaller peaks close to heptadecene and nonadecene that are suspected to be dienes. These are heptadecadiene at 13.85 minutes and nonadecadiene at 18.1 minutes (FIG. 3). Unfortunately, these species were present at too low a concentration to be identified by GC-MS, and no commercial diene standards were available with which to compare to their retention times. Their very low abundance suggests that the OleTje enzyme has a stronger preference for saturated fatty acids.

### Fatty Acid Feeding Increased Alkene Yield

As in the Rude et al. study, the inventors examined alkenes produced from yeast grown in media containing stearic acid (C18) or eicosanoic acid (C20) in addition to glucose. A three-fold increase in heptadecene was measured, with a two-fold increase in nonadecene when yeast were grown with stearic acid (FIG. 5). A three-fold increase in heptadecene was also found, with a five-fold increase in nonadecene when yeast are grown with eicosanoic acid. The greater increase in nonadecene further supports the contention that the OleTje enzyme has preference for the C20 fatty acid.

The inventors also looked at 5368 grown in media containing palmitic acid (C16) and oleic acid (C18:1). Palmitic acid had no effect on the alkene yields; in contrast to feeding Jeotgalicoccus with palmitic acid (Rude et al., as above) no pentadecene was observed in yeast cell extracts (data not shown). Oleic acid also had no impact on alkene yields; however, there was an increase in the GC peak at 13.85 minutes, suggesting an increase in the predicted heptadecadiene product (data not shown).

### oleTje Overexpression Increased Product Yield

As described above, strains were constructed with multiple copies of oleTje, with the expectation that higher production of the enzyme would lead to greater alkene yields. Yeast strains 5364 and 5365 carrying pS362 were also constructed (FIG. 2B, Table 2), with methionine-regulatable expression of oleTje, as well as strains 5366 and 5367 carrying pS363 (Table 2) with constitutive and strong expression of oleTje. The reason for using multiple strains, each an independent transformant of the oleTje 2μ plasmid, is that plasmid copy number can be variable between transformants, and so several were screened for maximal alkene production.

Alkene production of strains S364 and S365 grown with 0.4mM methionine after 3 days was examined (FIG. 6). Relative to the single-copy oleTje strain S368, S364 showed a modest increase in heptadecene (12%, 1.6 μg/L) and nonadecene (35%, 9 μg/L). S365 was more productive, yielding >4-fold more heptadecene (6.2 μg/L) and >2-fold more nonadecene (17.9 μg/L) than S368. Additionally, S365 was able to produce pentadecene (C15) at 1.6 μg/L. Lowering the methionine concentration to 0.2 mM (to fully derepress oleTje expression) further increased production of all three alkenes to a total titre of 31.6 μg/L, almost 4-fold higher than that achieved by S368.

Alkene production was next screened in strains 5366 and 5367, which have multiple copies of oleTje under the control of the strong constitutive PGK1 promoter (FIG. 6). Both strains were able to produce alkenes from C15 to C19 at levels well above 5368. The more productive strain is S367, likely due to more copies of the 2μ plasmid, which had a total alkene titre of 35 μg/L or >4-fold higher than that of S368. Total alkene produced, adjusted to dry cell weight, was 0.72 μg/g (S364), 1.3-2.2 μg/g (S365, more usually 1.3-1.6 μg/g), 2.1 μg/g (S366) and 3.5 μg/g (S367).

Based on these experiments, strain S365 was selected, whose oleTje expression is regulated by methionine, as the strain on which to base further metabolic engineering. Although strain S367 produced a slightly higher titre of alkenes, S365 gave the advantage of modulating oleTje expression, which was necessary when high enzyme activity leads to poor growth in combination with other genetic modifications.

### Genetic Engineering Towards Greater Alkene Production

The aim was to engineer a yeast strain that would generate as much alkene from glucose as possible. The metabolic capability of yeast has been investigated using genome-wide metabolic model iIN800 (Nookaew et al. (2008) BMC Syst. Biol. 2:71; doi:10.1186/1752-0509-2-71). The net stoichiometric equations for production of heptadecene and nonadecene from glucose are as follows:

9 Glucose+2 O₂+35 ATP+16 NADPH+18 NAD⁺−>2 Heptadecene+18 NADH+20 CO₂+18H₂O+2H⁺+16 NADP⁺+35 ADP+35 Pᵢ

10 Glucose+2 O₂+39 ATP+18 NADPH+20 NAD⁺−>2 Nonadecene+20 NADH+22 CO₂+20H₂O+2H⁺+18 NADP⁺+39 ADP+39 Pᵢ

Using both metabolic modelling and literature searching, a set of mutations was identified that could improve alkene yield by: (1) increasing the cytosolic concentration of NADPH; (2) increasing the intracellular concentration of oxygen by deleting the mitochondrial dehydrogenase gene MTD1; (3) reducing the carbon flux towards ethanol production through deletion of the alcohol dehydrogenase gene ADH1; (4) increasing the total free fatty acid pool through deletion of two genes encoding fatty acyl-CoA synthetases FAA1 and FAA4; and (5) increasing the C20 free fatty acid pool through deletion of ELO3 which encodes a fatty-acid elongase (FIG. 8).

Deletions at steps (2)-(5) were carried out in both the single-copy oleTje strain S368, as well as the oleTje overexpressing strain S365 (described in Materials and Methods).

### NADPH-Producing Enzymes Do Not Increase Alkene Yield

NADPH is a cofactor in over 50 reactions that are part of fatty acid synthesis and lipid metabolism, while NADH is involved in 8 fatty acid degradation reactions (iIN800, Nookaew et al. (2008), as above). Moreover, eight and nine molecules of NADPH are needed to synthesize one molecule of heptadecene and nonadecene, respectively. Therefore, the inventors examined whether increasing the NADPH/NADH ratio would lead to a higher concentration of free fatty acids, which would subsequently elevate alkene yield. In budding yeast, the majority of NADPH is synthesized in the mitochondria. Cytoplasmic NADPH, once utilised and converted to NADP, cannot be reformed in the cytoplasm. This capability was established in strain 5365 by introducing two genes encoding enzymes that produce NADPH from NADH.

The first gene encodes the soluble udhA transhydrogenase from E. coli, which interconverts NADH and NADPH. This has been used previously in yeast to either increase cytoplasmic NADPH or reduce NADH (Hou et al. (2009) Metabolic Engineering vol. 11 p 253-261; Toivari et al. (2010) Appl. Microbiol. & Biotechnol. vol 88 p 751-760). The second is a yeast gene, POS5, encoding a mitochondrial NADH kinase that uses ATP to phosphorylate NADH, generating NADPH. A truncated version of POS5 was used, without the mitochondrial targeting sequence; this version of the enzyme is maintained in the cytoplasm (Hou et al., 2009). Both genes were expressed from multicopy plasmids under the control of the strong PGK1 promoter; these plasmids also have the URA3 gene as a selectable marker for growth on media without uracil (plasmids pS263 and pS329, Table 2).

Metabolic modelling predicted modest increases in metabolic efficiency for production in alkenes in strains transformed with udhA and cytosolic POS5, at 3% and 2%, respectively. Experimentally, two independent transformants carrying the udhA plasmid were screened, strains 5372 and 5373 (FIG. 7). Both strains had a smaller titre of all three alkenes, C15, C17 and C19, relative to the S365 strain. In the Hou et al. study mentioned above, udhA expression had the effect of increasing both glycerol and ethanol production, and the increased flux through these competing pathways could explain the decreased alkene yield. Two independent transformants bearing the cytoplasmic POS5 plasmid, strains 5376 and 5377 were also screened (FIG. 7). Strain S377 had a 45% decrease in total alkene production but S376 produced 37% more alkenes relative to the untransformed S365 strain. The difference between the two POS5-carrying strains could be due to different copy numbers. Total alkene produced, adjusted to dry cell weight, was 1.6 μg/g (S372), 1.4 μg/g (S373), 2.7 μg/g (S376) 1.1 μg/g (S377).

Thus, by perturbing NADPH and NADH concentrations, at best a 37% increase in alkene production was achieved by expressing a cytoplasmic version of the Pos5 enzyme.

### The mtd1 Deletion Has A Modest Effect On Alkene Yield

The OleTje enzyme requires activated oxygen as a source of electrons for the decarboxylation of fatty acid to alkene (Rude et al. (2011) Appl. Environ. Microbiol. Vol. 77 p 1718-1727). Through metabolic modelling using the model iIN800 (Nookaew et al. (2008) BMC Syst. Biol. 2:71; doi:10.1186/1752-0509-2-71), the inventors identified the mtd1 deletion as a modification that would increase NADPH and oxygen availability for the OleTje reaction. Simulations have predicted that mtd1 deletion mutant can produce 10 times more nonadecene at the late exponential growth phase, without having any growth defects when compared to the parental strain.

MTD1 encodes a mitochondrial dehydrogenase (West et al, (1993) J. Biol. Chem. vol. 268 p 153-160), whose deletion leads to reduced oxidative phosphorylation within mitochondria. In the computer simulations, this resulted in greater oxygen availability for the OleTje reaction and consequently higher levels of alkenes. Mtd1p catalyses the cytosolic NAD-dependent conversion of 5,10-methylene-THF to 5,10-methenyl-THF, which is an essential reaction in the folate metabolism pathway. In the absence of Mtd1p, an NAD-dependent isoenzyme Ade3p catalyses the same conversion in the cytosol, which is expected to lead to a lower NADH/NAD+ ratio and make less NADH available to respirative metabolism, and eventually to lower respiration capacity with reduced utilization of oxygen. Experimentally, the mtd1 strain showed increased nonadecene production in the single copy oleTje strain background, while heptadecene was reduced (FIG. 9A). In the oleTje overexpression strain, the mtd1 deletion led to slightly reduced levels of pentadecene and nonadecene; however, heptadecene showed a modest increase (FIG. 9B). Based on the modelling predictions, alkene production of both mtd1 strains was also tested, in media supplemented with stearate. However, there was no significant increase in total alkene titres relative to the control strains (data not shown) (total alkene approximately 1.5 μg/g dry cell weight).

In the inventors' metabolic model, regulatory events are not represented, hence the redox balance of the cells may have shifted to other parts of the metabolism via more complicated routes than the present stoichiometric models can predict (see, for example, Bro et al. (2004) Biotechnology and Bioengineering, 85(3), 269-276. doi:10.1002/bit.10899 and Jouhten et al. (2008) BMC Systems Biology, 2, 60. doi:10.1186/1752-0509-2-60). This does not exclude the possibility that the mtd1 deletion mutant has the metabolic capability of higher alkene yields under well-aerated growth conditions, at higher levels of aeration than were tested experimentally here.

### The adh1 Deletion Increases the Alkene Yield Relative To Biomass

For S. cerevisiae, ethanol is the main product of glucose fermentation. This is true even under aerobic conditions if glucose concentrations are high. Therefore, fermentation is the key competing pathway to alkene production. The last step in ethanol biosynthesis is the reduction of acetaldehyde to ethanol, catalyzed by five yeast Adh enzymes, of which Adh1p plays the major role (reviewed by Leskovac et al. (2002) FEMS Yeast Res. vol. 2 p 481-494). In a previous study, an adh1 deletion was used successfully to reduce ethanol production and enhance the production of another metabolite in an engineered yeast strain (Tokuhiro et al. (2009) Appl. Microbiol. & Biotechnol. vol. 82 p 883-890). The inventors reasoned that deleting ADH1 in the oleTje strains could also lead to increased production of alkenes. This was supported by simulations made using model iIN800, where blocking ethanol production re-directed the carbon flux to other products including alkenes.

The ADH1 gene was disrupted in the oleTje single-copy strain, 5368. The resulting strain, 5434, is very-slow growing, probably because of the accumulation of acetaldehyde which is toxic to yeast (Marisco et al. (2011) Yeast vol. 28 p 363-373; Tokuhiro et al. (2009), as above). This strain produced only half the total alkene yield of S368 (FIG. 8A); however, it was able to produce the C15 alkene, pentadecene, which had not previously been possible without oleTje overexpression. Additionally, when alkene production was normalised to biomass, the adh1 strain generated twice the total alkene yield of S368, at 1.3 μg/g dry cell weight (FIG. 10A). These results suggest that increasing the growth rate of the adh1 strain, particularly by pushing acetaldehyde utilisation towards fatty acid biosynthesis, could lead to higher alkene production.

### Increasing the Total Fatty Acid Pool Boosts Heptadecene

Through the yeast modelling, the inventors identified a list of enzymes that directly act on the free fatty acid pool (FIG. 8). Of particular interest were the family of fatty acid activating enzymes (Fat1 and Faa1-4), which utilize fatty acid and CoAs to synthesize fatty acyl-CoA (reviewed in Tehlivets et al. (2007) Biochim et Biophys. Acta. vol. 1771 p 2765-2778). Scharnewski et al. (2008; FEBS J. vol. 275 p 2765-2778) have shown that the double deletion, faa1 faa4, leads to elevation of free fatty acids in the yeast cell (approximately 50-fold) and even secretion of fatty acids into the medium. Therefore, the effect of this double deletion on alkene production in the oleTje strains was tested.

The result of the Scharnewski et al. study was confirmed, that faa1 faa4 strains secrete fatty acid into the culture medium, as evidenced by white precipitates. In the cell lysates, an increase in both saturated and unsaturated C16 and C18 fatty acids and their corresponding ethyl esters was also measured (data not shown). This double mutation also led to a several-fold increase in alkene yield, particularly in heptadecene, in both the 5368 (FIG. 9A) and 5365 (FIG. 9B) background strains. The increase heptadecene yield was over ten-fold, although this was at the expense of a slight reduction in nonadecene production. Interestingly, no significant increase in the pentadecene yield was detected, even though palmitic acid (C16) appeared to be as highly elevated as stearic acid (C18) in the strains. This finding is consistent with the results of the palmitic acid feeding experiment, and confirms the greater preference of OleTje enzyme for stearic acid over palmitic acid. For strain 5437, which combined oleTje overexpression with the faa1 faa4 double mutation, total alkene yield was 96 μg/L, nearly ten-fold greater than that of our initial strain, S368 (FIG. 9B). This corresponds to 6-6.5 μg alkene produced for every gram of dry cell weight, also ten-fold that obtained with S368 (FIG. 10B).

### Increasing Cellular C20 Fatty Acid Boosts Nonadecene

Because the most preferred substrate for OleTje is eicosanoic acid (C20), the inventors searched for a mutation that would increase this particular fatty acid pool. ELO3 was identified, which encodes a long-chain acyl elongase, responsible for the synthesis of very long chain fatty acids ranging from C20 to C26 (Oh et al. (1997) J. Biol. Chem. vol. 272 p 17376-17384). The elo3 null mutant is viable because its activity partially overlaps with another elongase-coding gene, ELO2. However, this mutant shows pleiotropic phenotypes and an altered membrane and lipid profile (Oh et al. (1997), as above; Ejsing et al. (2009) Proc. Natl. Acad. Sci. U.S.A. vol. 106 p 2136-2141). Eicosanoic acid (C20) is elevated, in the elo3 mutant, to a level 5-fold greater than that found in the parental strain (Oh et al. (1997), as above).

The elo3 mutation led to poor growth in both oleTje strain backgrounds. The oleTje overexpression strain was particularly affected, yielding only one third the biomass of the background strain, S365. This engineered yeast, 5431, displayed the large and elongated cell morphology that is characteristic of yeast undergoing cellular stress (data not shown). Despite this, the elo3 strains produced alkenes at a several-fold greater yield than in background strains, due to elevated nonadecene (FIGS. 9A and 9B). For strain 5431, which combined oleTje overexpression with elo3 deletion, the total alkene yield was 116 μp g/L, which is a >10-fold increase over that of the initial strain, S368 (FIG. 9B). When normalised for biomass, 5431 produced 23 μg of alkenes per gram of dry cell weight, corresponding to a 40-fold increase over the initial strain. This suggests a potential for increasing alkene yield by improving strain biomass; in particular, by turning off ELO3 expression only when the culture has established a particular cell density.

### EXAMPLES

### Discussion

The inventors demonstrated production of terminal alkenes in S. cerevisiae through the activity of a fatty acid decarboxylase, OleTje, from the bacterium, Jeotgalicoccus sp. This enzyme has a high preference for eicosanoic acid (C20), followed by stearic acid (C18) and very low activity towards palmitic acid (C16). In contrast to in vitro assays (Rude et al (2011) Appl. Eviron. Microbiol. vol. 77 p 1718-1727), activity towards myristic acid (C14) was not detected, although it is more abundant than eicosanoic acid (Oh et al. (1997) J. Biol. Chem. vol. 272 p 17376-17384). This was probably because it is a poor OleTje substrate within the yeast cell.

OleTje activity was observed through overexpression and in combination with specific modifications in yeast fatty acid metabolism. The results suggest that alkene production in budding yeast could be further optimised through a variety of methods.

First, the highest possible oleTje copy number using the 2μ plasmid has not been achieved. Multicopy oleTje strains 5365 and 5367 (FIGS. 1 and 6) grow as robustly as the control strain YSBN8, suggesting that yeast would tolerate higher copy numbers of the heterologous gene. An alternative strategy to the LEU2 2μ plasmid, which is able to complement leucine auxotrophy at a copy number of 1, is to use the weaker leu2d allele as the selectable plasmid marker. This has been shown to produce copy numbers of over 100 because of its poor ability to complement leucine auxotrophy (Moriya et al. (2006) Plos Genetics 2(7), e111). For convenience and gene stability, multiple copies of oleTje could also be integrated into the yeast genome at the ribosomal DNA locus using the leu2d selectable marker.

Improving initial strain biomass, particularly for the adh1 and elo3 alkene-producing strains, could also enhance the alkene yield. This may be achieved by regulated depletion of the Adh1 and Elo3 gene products through use of a repressible promoter such as tetO2 (Garí et al. (1997) Yeast vol. 13 p 837-848).

In yeast, approximately 70% of the free fatty acid pool is comprised of the monounsaturated acids palmitoleate (C16) and oleate (C18), each several hundred-fold more abundant than eicosanoic acid (Oh et al. (1997) J. Biol. Chem. vol. 272 p 17376-17384). Therefore, the greatest improvement in alkene yield could be gained from engineering OleTje specificity towards these highly abundant fatty acids. This can be achieved either by random mutagenesis of its characterised active site (Rude et al. (2011) Appl. Environ. Microbiol. vol 77 p 1718-1727) or directed mutagenesis based on sequence comparison with related fatty acid decarboxylases/hydroxylases of known substrate specificity.

The inventors have demonstrated alkene production in S. cerevisiae, which was enhanced with minimal modifications to its lipid metabolism. This is an ideal environment for the initial study as the computational and experimental tools available enable a fast pace at which strain engineering and information can be obtained. However, this is not the optimum background for alkene production because of its limited lipid content and tendency to make ethanol. This study brings about the exciting prospect of using the fatty acid decarboxylase approach to produce alkenes from oleaginous yeasts. This group of yeasts (reviewed in Beopoulos et al. (2011; Appl. Microbiol. Biotechnol. vol. 90 p 1193-1206) and Sabirova et al. (2011; Microbial Biotechnol. vol. 4 p 47-54) assimilate lipid and are currently being developed as factories for biofuels.

TABLE 1
Yeast Strains Used in this Study

| | | | Total alkene |
|---|---|---|---|
| | | | yield |
| Strain | | | (μg/g dry cell |
| Name | Genotype | Reference | mass) |
| FY2 | MATα ura3-52 | Winston et al., | 0 |
| | | 1995 | |
| YSBN8 | MATα ura3-52 hoΔ::hph | Canelas et al., | 0 |
| | | 2010 | |
| S240 | MATα ura3-52 leu2Δ::PGKp-oleTje::hph | this study | not measured |
| S364 | MATα ura3-52 leu2Δ::PGKp-oleTje::hph + | this study | 0.72 |
| | plasmid S362 2μ(LEU2, MET25p- | | |
| | oleTje) | | |
| S365 | MATα ura3-52 leu2Δ::PGKp-oleTje::hph + | this study | (1.3-2.2) |
| | plasmid S362 2μ(LEU2, MET25p- | | Typically: |
| | oleTje) | | 1.3-1.6 |
| S366 | MATα ura3-52 leu2Δ::PGKp-oleTje::hph + | this study | 2.1 |
| | plasmid S362 2μ(LEU2, PGKp-oleTje) | | |
| S367 | MATα ura3-52 leu2Δ::PGKp-oleTje::hph + | this study | 3.5 |
| | plasmid S362 2μ(LEU2, PGKp-oleTje) | | |
| S368 | MATα ura3-52 leu2Δ::PGKp-oleTje::hph + | this study | 0.3 to 0.6 |
| | plasmid pRS425 2μ(LEU2) | | Typically: 0.5-0.6 |
| S372 | MATα ura3-52 leu2Δ::PGKp-oleTje::hph + | this study | 1.6 |
| | plasmid S362 2μ(LEU2, MET25p- | | |
| | oleTje) + plasmid S263 2μ(URA3, PGK1p- | | |
| | udhA) | | |
| S373 | MATα ura3-52 leu2Δ::PGKp-oleTje::hph + | this study | 1.4 |
| | plasmid S362 2μ(LEU2, MET25p- | | |
| | oleTje) + plasmid S263 2μ(URA3, PGK1p- | | |
| | udhA) | | |
| S376 | MATα ura3-52 leu2Δ::PGKp-oleTje::hph + | this study | 2.7 |
| | plasmid S362 2μ(LEU2, MET25p- | | |
| | oleTje) + plasmid S329 2μ(URA3, PGK1p- | | |
| | cytoplasmic POS5) | | |
| S377 | MATα ura3-52 leu2Δ::PGKp-oleTje::hph + | this study | 1.1 |
| | plasmid S362 2μ(LEU2, MET25p- | | |
| | oleTje) + plasmid S329 2μ(URA3, PGK1p- | | |
| | cytoplasmic POS5) | | |
| S391 | MATα ura3-52 leu2Δ::PGK1p-oleTje::hph | this study | 1.9 |
| | faa1Δ::kanMX4 faa4Δ::natMX4 + plasmid | | |
| | pRS425 [2μ, LEU2] | | |
| S392 | MATα ura3-52 leu2Δ::PGK1p-oleTje::hph | this study | 4.6 |
| | mtd1Δ::kanMX4 + plasmid pRS425 [2μ, | | |
| | LEU2] | | |
| S431 | MATα ura3-52 leu2Δ::PGKp-oleTje::hph | this study | 23 |
| | elo3Δ::kanMX4 + plasmid S362 2μ(LEU2, | | |
| | MET25p-oleTje) | | |
| S432 | MATα ura3-52 leu2Δ::PGKp-oleTje::hph | this study | 1.8 |
| | elo3Δ::kanMX4 + plasmid pRS425 | | |
| | 2μ(LEU2) | | |
| S434 | MATα ura3-52 leu2::PGKp-oleTje::hph | this study | 1.3 |
| | adh1Δ::kanMX4 + plasmid pRS425 (2μ, | | |
| | LEU2) | | |
| S437 | MATα ura3-52 leu2Δ::PGKp-oleTje::hph | this study | 6-6.5 |
| | faa1Δ::kanMX4 faa4Δ::natMX + plasmid | | |
| | S362 2μ(LEU2, MET25p-oleTje) | | |
| S439 | MATα ura3-52 leu2Δ::PGKp-oleTje::hph | this study | 1.5 |
| | mt d1Δ::kanMX4 + plasmid S362 | | |
| | 2μ(LEU2, MET25p-oleTje) | | |

TABLE 2
Plasmids Used in this Study

| Plasmid | | |
|---|---|---|
| Name | Description | Reference |
| pRS425 | 2μ, LEU2 | Sikorski |
| | | and Hieter, |
| | | 1989 |
| pS239 | pMS (GeneArt) with oleTje integration | this study |
| (SEQ | construct flanked by LEU2 homologous | |
| ID NO: 5) | sequences (NotI to SpeI) | |
| pS263 | pRS426 (XhoI BamHI) + udhA with | this study |
| (SEQ | PGK promoter and CycT terminator (XhoI to | |
| ID NO: 6) | BglII) | |
| pS329 | pRS426 (XhoI BamHI) + cytoplasmic | this study |
| (SEQ | POS5 with PGK promoter and CycT terminator | |
| ID NO: 7) | (XhoI to BglII) | |
| pS362 | pRS425 with strep-tagged oleTje gene | this study |
| (SEQ | under control of the MET25 promoter and | |
| ID NO: 3) | Cyc1 terminator | |
| pS363 | pRS425 with strep-tagged oleTje gene | this study |
| (SEQ | under control of the PGK1 promoter and Cyc1 | |
| ID NO: 4) | terminator | |

TABLE 3
DNA Primers Used in this Study

| | | | |
|---|---|---|---|
| Name | Sequence | Description | Reference |

LEU2 5′ Primer Mix (for S240 creation)

| | | | |
|---|---|---|---|
| leu2-C1-F | TGGGAGAAAAAGGAAA | Forward primer for both | this study |
| | GGTG (SEQ ID NO: 8) | reactions below | |
| PGK-R | TGCAGGTATGCGATAGT | Reverse primer producing | this study |
| | TCC | 290 bp DNA with correct | |
| | (SEQ ID NO: 9) | integration at LEU2 locus | |
| leu2-C-noins- | TCGGATGCAAAGTTACA | Negative control reverse | this study |
| R | TGG | primer producing 565 bp | |
| | (SEQ ID NO: 10) | DNA when there is no | |
| | | integration at LEU2 site | |

LEU2 3′Primer Mix (for S240)

| leu2-C1-R | TGCCCTCCTCCTTGTCAA | Both primers produce 638 bp | this study |
|---|---|---|---|
| | TA | DNA when the construct is | |
| | (SEQ ID NO: 11) | correctly integrated | |
| hph-F | ACTGTCGGGCGTACACA | | |
| | AAT | | |
| | (SEQ ID NO: 12) | | |
| NADPH Producing Enzymes | | | |
| udhA-Nco-F | agccatggtATGCCACATTCC | to amplify udhA gene from | this study; Hou et |
| | TACGATTACGATGCC | E. coli K12 JM109 strain | al., 2009 |
| | (SEQ ID NO: 13) | | |
| udhA-R | gtggatccTTAAAACAGGCG | | this study; Hou et |
| | Gtttaaac (SEQ ID NO: 14) | | al., 2009 |
| cytPos5-Nde- | ctccatATGAGTACGTTGG | to amplify the truncated | this study; Hou et |
| F | ATTCACATTC (SEQ ID | POS5 gene from yeast | al., 2009 |
| | NO: 15) | genomic DNA | |
| Pos5-R | gaggatccTTAATCATTATC | | this study; Hou et |
| | AGTCTGTCTCTTGGTC | | al., 2009 |
| | (SEQ ID NO: 16) | | |
| Gene Deletions | | | |
| kanB | CTGCAGCGAGGAGCCGT | SGD page; internal to | Saccharomyces |
| | AAT | kanMX; R near 5′ | Genome Deletion |
| | (SEQ ID NO: 17) | | Project |
| kanC | TGATTTTGATGACGAGC | SGD page; internal to | Saccharomyces |
| | GTAAT (SEQ ID NO: 18) | kanMX; R near 3′ | Genome Deletion |
| | | | Project |
| natB | GGTGAAGGACCCATCCA | >100 bp from start of | this study |
| | GT (SEQ ID NO: 19) | nat1MX ORF | |
| natC | gGATGGGGTTCACCCTCT | 100 bp from end of nat1MX | this study |
| | G (SEQ ID NO: 20) | ORF | |
| adh1-koF | ATACGGCCTTCCTTCCA | used to amplify knockout | this study |
| | GTT (SEQ ID NO: 21) | construct from | |
| adh1-koR | GATTTGCAGGCATTTGC | adh1Δ::kanMX4 strain | this study |
| | TC (SEQ ID NO: 22) | | |
| adh1-A | CTTCATTCACGCACACT | to verify adh1 knockout, 5′ | Saccharomyces |
| | ACTCTCTA (SEQ ID | of gene | Genome Deletion |
| | NO: 23) | | Project |
| adh1-B | ACTAATGGTAGCTTAAC | negative control reverse | Saccharomyces |
| | TGGCAATG (SEQ ID | primer for ADH1 | Genome Deletion |
| | NO: 24) | Project | |
| adh1-D | GGACATAAAATACACAC | to verify adh1 knockout, 3′ | Saccharomyces |
| | CGAGATTC (SEQ ID | of gene | Genome Deletion |
| | NO: 25) | | Project |
| mtd1-ko-F | CCTTGCAACCAGCTTCTT | used to amplify knockout | this study |
| | CTT (SEQ ID NO: 26) | construct from | |
| mtd1-ko-R | TCGTATTCGGATGATGG | mtd1Δ::kanMX4 strain | |
| | ACAA (SEQ ID NO: 27) | | |
| mtd1-A | TATTCTTTTCCTAGTCCA | to verify mtd1 knockout, 5′ | Saccharomyces |
| | AGTCCTG (SEQ ID NO: 28) | of gene | Genome Deletion |
| | | | Project |

TABLE 4
GC Standards Used and Retention Times

| | | |
|---|---|---|
| Name | Carbon length | Retention time |

Internal Standards

| | | |
|---|---|---|
| Ethyl heptanoate | C7 ethyl ester | 4.852 |
| Tetradecane | C14:0 alkane | 6.276 |
| 1-Tetradecene | C14:1 alkene | 6.992 |
| Pentadecanol | C15 alcohol | 22.914 |

Alkenes

| | | |
|---|---|---|
| 1-Pentadecene | C15:1 | 9.090 |
| 1-Heptadecene | C17:1 | 13.553 |

Ethyl Esters

| | | |
|---|---|---|
| Ethyl butyrate | C4 | 2.124 |
| Ethyl hexanoate | C6 | 3.570 |
| Ethyl caprylate | C8 | 6.611 |
| Ethyl decanoate | C10 | 10.840 |
| Ethyl dodecanoate | C12 | 15.332 |
| Ethyl myristate | C14 | 19.540 |
| Ethyl palmitate | C16 | 22.784 |
| Ethyl stearate | C18 | 25.450 |
| Ethyl eicosanoate | C20 | 27.781 |
| Ethyl behenate | C22 | 29.919 |
| Ethyl tetracosanoate | C24 | 31.600 |

Fatty Acids

| | | |
|---|---|---|
| Palmitic acid | C16:0 | 28.35 |
| Palmitoleic acid | C16:1 | 28.577 |
| Stearic acid | C18:0 | 30.26 |
| Oleic acid | C18:1 | 30.554 |

1. An alkene-producing yeast cell comprising a bacterial fatty acid decarboxylase enzyme.

2. The cell of claim 1 wherein the bacterial fatty acid decarboxylase enzyme comprises the amino acid sequence shown in SEQ ID NO:1.

3. The cell of claim 1 further comprising a polynucleotide encoding the bacterial fatty acid decarboxylase enzyme.

4. The cell of claim 3 wherein the polynucleotide comprises the nucleic acid sequence shown in SEQ ID NO:2.

5. The cell of claim 3 having a genome comprising the polynucleotide.

6. The cell of claim 1, wherein the cell is a Saccharomyces cell.

7. The cell of claim 1, wherein the cell is an oleaginous yeast cell.

8. The cell of claim 7 wherein the oleaginous yeast cell is a member of a genus selected from the group consisting of Yarrowia, Candida, Rhodotorula, Rhodosporidium, Cryptococcus, Trichosporon, and Lipomyces.

9. The cell of claim 8, wherein the cell is of the species selected from the group consisting of Lipomyces starkeyi, Rhodosporidium toruloides, Rhodotorula glutinis, and Yarrowia lipolytica.

10. The cell of claim 1 further comprising at least one copy of a plasmid having nucleotide sequence SEQ ID NO:3 or 4 or a functional variant or portion thereof.

11. The cell of claim 1 wherein the alkene produced by the cell is a terminal alkene.

12. The cell of claim 1 wherein the alkene produced by the cell has 15, 16, 17, 18 or 19 carbon atoms.

13. The cell of claim 1 further comprising a modification to provide an internal free fatty acid pool greater than the pool in a non-modified equivalent cell.

14. The cell of claim 13 wherein the modification comprises a disruption of an elo3 gene or a homologous gene thereof.

15. The cell of claim 13 wherein the modification comprises disruption of a faa1 and a faa4 gene or a homologous gene thereof.

16. The cell of claim 1 further comprising a modification to disrupt conversion of acetaldehyde to ethanol by the cell.

17. The cell of claim 16 wherein the modification comprises a disruption of an adh gene or a homologous gene thereof.

18. The cell of claim 1 further comprising a modification to provide an internal oxygen concentration greater than the concentration in a non-modified equivalent cell.

19. The cell of claim 18 wherein the modification comprises a disruption of a mitochondrial dehydrogenase-encoding gene.

20. A method of preparing an alkene comprising culturing an alkene-producing yeast cell comprising a bacterial fatty acid decarboxylase enzyme.

21. The method of claim 20 wherein the cell is cultured in a medium comprising at least one fatty acid.

22. The method of claim 21 wherein the fatty acid comprises 16, 17, 18, 19 or 20 carbon atoms.

23. The method of claim 20 further comprising isolating an alkene from the cell by a cell lysis method which does not include use of a detergent and a reducing agent.

24. The method of claim 23 wherein the reducing agent is tris(hydroxypropyl)phosphine.

25. The method of claims 23 comprising lysing the cell by agitation with glass beads.
